(19) United States
(12) Patent Application Publication
(10) Pub. No.: US 2014/0086825 A1
(43) Pub. Date: Mar. 27, 2014

(54) METHODS AND KITS FOR ASSESSING, MODULATING AND TREATING SUBSTANCES AFFECTING BODILY PROCESSES

(71) Applicant: William J. McFaul, (US)

(72) Inventor: William J. McFaul, Jackson, NJ (US)

(21) Appl. No.: 14/033,554

(22) Filed: Sep. 23, 2013

Related U.S. Application Data
(60) Provisional application No. 61/704,830, filed on Sep. 24, 2012.

(51) Int. Cl. G01N 33/50

(52) U.S. Cl. CPC G01N 33/50
USPC 424/1.11; 424/722

(57) ABSTRACT

Methods, systems, kits and devices for assessing and providing for homeostasis of signaling molecules in an individual via assessing, modulating and treating levels of modulators are provided.

This patent application claims the benefit of priority from U.S. Provisional Application Ser. No. 61/704,830, filed Sep. 24, 2013, the teachings of which are herein incorporated by reference in their entirety.

### FIELD OF THE INVENTION

The present invention relates to the field of identifying, testing for, and modulating corollary relationships between bodily substances and their interactions. In particular, the present invention is directed to the method and kits for identifying, testing for and modulating signaling molecules in the body of humans and animals.

### BACKGROUND

All forms of life are supported by a variety of substances; including but not limited to hormones, proteins, peptides, amino acids, elements (minerals), protein kinases, vitamins (chemicals or compounds that function as signaling molecules) and bacteria. These substances create the homeostasis essential to maintain life for humans as well as plants and animals. It should be noted that many hormones and proteins are signaling molecules.

Associated substances affect levels of each other in the body. Additionally, certain other substances cooperate with the levels of the associated substances. Furthermore, certain substances are a part of more than one group that affects the substances. These relationships can be utilized to determine various health parameters of an individual or animal; however, to date, their corollary relationships have not been established in order to effectively use them.

Therefore, there is a need in the field to identify and assess the homeostatic relationship between the substances that are important to the body's functioning via the measuring of various levels of the substances that constitute the associated substances. In lay terms, if a bodily substance (e.g. a signaling molecule) were comprised of eggs, flour and milk; the levels of each would determine the outcome (efficacy). Numerous factors can influence the outcomes; examples include size of the eggs, altitude, freshness and type of the milk, etc. If the quantities of each constituent are not within a particular range, outcomes will be impacted. These same scenarios apply to the body's signaling molecules. Current methods do not exist for the assessment of current levels of each existing micronutrients that will establish the need for replenishment of essential substances such as particular amino acids or elements (e.g. those listed on the Period Chart). There is also a need to utilize the homeostatic relationships in order to develop methods and systems for promoting the formation of personalized therapeutic strategies to ensure wellness; i.e. a healthy individual or animal.

### SUMMARY OF THE INVENTION

An aspect of the invention is to identify the specific substances that must be in balance (homeostasis) in promoting the formation of secondary substances; e.g. signaling molecules.

Another aspect of the invention is a method of establishing tests to determine imbalances between the relationships of substances that promote the formation of secondary substances; e.g. signaling molecules thereby affecting bodily processes.

Another aspect of the invention is to determine the interactions between substances that promote formation of secondary substance; e.g. signaling molecules and the outcomes when signaling molecules are not adequately formed to facilitate their interactions.

Another aspect of the invention is a method and kit for assessing, modulating and treating existing levels of the constituents necessary for the signaling molecule Tight Junction Protein 1 comprising: measuring a level of calcium; measuring a level of magnesium; measuring a level of phenylalanine; comparing the measured levels; and selecting and/or administering when necessary a treatment based upon the measured levels.

Another aspect of the invention is a method and kit for assessing, modulating and treating existing levels of the constituents necessary to form the signaling molecule Tight Junction Protein 2 comprising: measuring a level of calcium; measuring a level of magnesium; measuring a level of tyrosine; comparing the measured levels; and means for establishing homeostasis of Tight Junction Protein 2 based upon the measured levels.

Another aspect of the invention is a method for assessing, modulating and/or treating existing levels of the constituents necessary to form the signaling molecule Tight Junction Protein 3 comprising: measuring a level of calcium; measuring a level of magnesium; measuring a level of tryptophan; comparing the measured levels; and selecting and/or administering when necessary a treatment based upon the measured levels.

Another aspect of the invention is a method and kit for assessing, modulating and/or treating existing levels of the constituents necessary to form the signaling molecule and neurohormone aldosterone comprising: measuring a level of sulfur; measuring a level of nitric oxide; measuring a level of iron; comparing the measured levels; and selecting and/or administering when necessary a treatment based upon the measured levels.

Another aspect of the invention is a method and kit for assessing, modulating and/or treating existing levels of the constituents necessary to form the signaling molecule and neurohormone oxytocin comprising: measuring a level of sulfur; measuring a level of arginine; measuring a level of iron; comparing the measured levels; and selecting and/or administering when necessary a treatment based upon the measured levels.

Another aspect of the invention is a method and kit for assessing, modulating and/or treating existing levels of the constituents necessary to form the signaling molecule and neurohormone serotonin comprising: measuring a level of sulfur; measuring a level of histidine; measuring a level of iron; comparing the measured levels; and selecting and/or administering when necessary a treatment based upon the measured levels.

Another aspect of the invention is a method and kit for assessing, modulating and/or treating existing levels of the constituents necessary to form the signaling molecule and neurohormone adrenaline comprising: measuring a level of zinc; measuring a level of tryptophan; measuring a level of nickel; comparing the measured levels; and selecting and/or administering when necessary a treatment based upon the measured levels.

Another aspect of the invention is a method and kit for assessing, modulating and/or treating existing levels of the constituents necessary to form the signaling molecule and neurohormone cortisol comprising: measuring a level of zinc; measuring a level of nitric oxide; measuring a level of nickel; comparing the measured levels; and selecting and/or administering when necessary a treatment based upon the measured levels.

Another aspect of the invention is a method and kit for assessing, modulating and/or treating existing levels of the constituents necessary to form the signaling molecule p53 comprising: measuring a level of iron; measuring a level of phosphorus; measuring a level of sulfur; comparing the measured levels; and selecting and/or administering when necessary a treatment based upon the measured levels.

Another aspect of the invention is a method and kit for assessing, modulating and/or treating existing levels of the constituents necessary to form the signaling molecule p63 comprising: measuring a level of copper; measuring a level of zinc; measuring a level of calcium; comparing the measured levels; and selecting and/or administering when necessary a treatment based upon the measured levels.

Another aspect of the present invention is a method and kit for assessing, modulating and/or treating the existing levels of the constituents necessary to form the signaling molecule p73 comprising: measuring a level of nickel; measuring a level of zinc; measuring a level of boron; comparing the measured levels; and selecting and/or administering when necessary a treatment based upon the measured levels.

Another aspect of the invention is a method and kit for assessing, modulating and/or treating existing levels of the constituents necessary to form the signaling molecule vitamin K2 comprising: measuring a level of phosphorus; measuring a level of leucine; measuring a level of calcium; comparing the measured levels; and selecting and/or administering when necessary a treatment based upon the measured levels.

Another aspect of the invention is a method and kit for assessing, modulating and/or treating existing levels of the constituents necessary to form the signaling molecule vitamin K3 (a.k.a. MK4) comprising: measuring a level of phosphorus; measuring a level of valine; measuring a level of calcium; comparing the measured levels; and selecting and/or administering when necessary a treatment based upon the measured levels.

Another aspect of the invention is a method and kit for assessing, modulating and/or treating existing levels of the constituents necessary to form the signaling molecule Interleukin 15 comprising: measuring a level of phosphorus; measuring a level of nitric oxide; measuring a level of calcium; comparing the measured levels; and selecting and/or administering when necessary a treatment based upon the measured levels.

Another aspect of the invention is a method and kit for assessing, modulating and/or treating existing levels of the constituents necessary to form the signaling molecule Interleukin 17 comprising: measuring a level of phosphorus; measuring a level of nitric oxide; measuring a level of chromium; comparing the measured levels; and selecting and/or administering when necessary a treatment based upon the measured levels.

Another aspect of the invention is a method and kit for assessing, modulating and/or treating existing levels of the constituents necessary to form the signaling molecule nitric oxide synthase 3 comprising: measuring a level of bromine; measuring a level of phenylalanine; measuring a level of iodine; comparing the measured levels; and selecting and/or administering when necessary a treatment based upon the measured levels.

Another aspect of the invention is a method and kit for assessing, modulating and/or treating existing levels of the constituents necessary to form the signaling molecule nitric oxide synthase 3 comprising: measuring a level of bromine; measuring a level of tyrosine; measuring a level of iodine; comparing the measured levels; and selecting and/or administering when necessary a treatment based upon the measured levels.

Another aspect of the invention is a method and kit for assessing, modulating and/or treating existing levels of the constituents necessary to form the signaling molecule nitric oxide synthase 3 comprising: measuring a level of bromine; measuring a level of tryptophan; measuring a level of iodine; comparing the measured levels; and selecting and/or administering when necessary a treatment based upon the measured levels.

Another aspect of the invention is a method and kit for assessing, modulating and/or treating existing levels of the constituents necessary to form the signaling molecule HMOX1 comprising: measuring a level of iron; measuring a level of threonine; measuring a level of sulfur; comparing the measured levels; and selecting and/or administering when necessary a treatment based upon the measured levels.

Another aspect of the invention is a method and kit for assessing, modulating and/or treating existing levels of the constituents necessary to form the signaling molecule HMOX2 comprising: measuring a level of iron; measuring a level of serine; measuring a level of sulfur; comparing the measured levels; and selecting and/or administering when necessary a treatment based upon the measured levels.

Another aspect of the invention is a method and kit for assessing, modulating and/or treating existing levels of the constituents necessary to form the signaling molecule HMOX3 comprising: measuring a level of iron; measuring a level of cysteine; measuring a level of sulfur; comparing the measured levels; and selecting and/or administering when necessary a treatment based upon the measured levels.

Another aspect of the invention is a method and kit for assessing, modulating and/or treating existing levels of the constituents necessary to form the signaling molecule TIG-1 comprising: measuring a level of phosphorus; measuring a level of phenylalanine; measuring a level of calcium; comparing the measured levels; and selecting and/or administering when necessary a treatment based upon the measured levels.

Another aspect of the invention is a method and kit for assessing, modulating and/or treating existing levels of the constituents necessary to form the signaling molecule TIG-2 comprising: measuring a level of phosphorus; measuring a level of tyrosine; measuring a level of calcium; comparing the measured levels; and selecting and/or administering when necessary a treatment based upon the measured levels.

Another aspect of the invention is a method and kit for assessing, modulating and/or treating existing levels of the constituents necessary to form the signaling molecule TIG-3 comprising: measuring a level of phosphorus; measuring a level of tryptophan; measuring a level of calcium; comparing the measured levels; and selecting and/or administering when necessary a treatment based upon the measured levels.

Another aspect of the invention is a method and kit for assessing, modulating and/or treating existing levels of the constituents necessary to form the signaling molecule HER-1 comprising: measuring a level of phosphorus; measuring a level of tyrosine; measuring a level of chromium; comparing the measured levels; and selecting and/or administering when necessary a treatment based upon the measured levels.

Another aspect of the invention is a method and kit for assessing, modulating and/or treating existing levels of the constituents necessary to form the signaling molecule HER-2 comprising: measuring a level of phosphorus; measuring a level of phenylalanine; measuring a level of chromium; comparing the measured levels; and selecting and/or administering when necessary a treatment based upon the measured levels.

Another aspect of the invention is a method and kit for assessing, modulating and/or treating existing levels of the constituents necessary to form the signaling molecule HER-3 comprising: measuring a level of phosphorus; measuring a level of tryptophan; measuring a level of chromium; comparing the measured levels; and selecting and/or administering when necessary a treatment based upon the measured levels.

Another aspect of the invention is a method and kit for assessing, modulating and/or treating existing levels of the constituents necessary to form the signaling molecule acetylcholinesterase comprising: measuring a level of phosphorus; measuring a level of histidine; measuring a level of calcium; comparing the measured levels; and selecting and/or administering when necessary a treatment based upon the measured levels.

Another aspect of the invention is a method and kit for assessing, modulating and/or treating existing levels of the constituents necessary to form the signaling molecule butylcholinesterase comprising: measuring a level of phosphorus; measuring a level of arginine; measuring a level of calcium; comparing the measured levels; and selecting and/or administering when necessary a treatment based upon the measured levels.

Another aspect of the invention is a method and kit for assessing, modulating and/or treating existing levels of the constituents necessary to form a signaling molecule for nucleotide activity comprising: measuring a level of phosphorus; measuring a level of sulfur; measuring a level of histidine; comparing the measured levels; and selecting and/or administering when necessary a treatment based upon the measured levels.

Another aspect of the invention is a method and kit for assessing, modulating and/or treating existing levels of the constituents necessary to form a signaling molecule for nucleotide activity comprising: measuring a level of phosphorus; measuring a level of sulfur; measuring a level of arginine; comparing the measured levels; and selecting and/or administering when necessary a treatment based upon the measured levels.

Another aspect of the invention is a method and kit for assessing, modulating and/or treating existing levels of the constituents necessary to form a signaling molecule for nucleotide activity comprising: measuring a level of phosphorus; measuring a level of silicon; measuring a level of histidine; comparing the measured levels; and selecting and/or administering when necessary a treatment based upon the measured levels.

Another aspect of the invention is a method and kit for assessing, modulating and/or treating existing levels of the constituents necessary to form a signaling molecule for nucleotide activity comprising: measuring a level of phosphorous; measuring a level of silicon; measuring a level of arginine; comparing the measured levels; and selecting and/or administering when necessary a treatment based upon the measured levels.

Another aspect of the invention is a method and kit for assessing, modulating and/or treating existing levels of the constituents necessary to form a signaling molecule for nucleotide activity comprising: measuring a level of phosphorous; measuring a level of silicon; measuring a level of lysine; comparing the measured levels; and selecting and/or administering when necessary a treatment based upon the measured levels.

Another aspect of the invention is a method and kit for assessing, modulating and/or treating existing levels of the constituents that form a signaling molecule for nucleotide activity comprising: measuring a level of phosphorus; measuring a level of silicon; measuring a level of threonine; comparing the measured levels; and selecting and/or administering when necessary a treatment based upon the measured levels.

Another aspect of the invention is a method and kit for assessing, modulating and/or treating existing levels of the constituents that form another signaling molecule for nucleotide activity comprising: measuring a level of phosphorus; measuring a level of silicon; measuring a level serine; comparing the measured levels; and selecting and/or administering when necessary a treatment based upon the measured levels.

Another aspect of the invention is a method and kit for assessing, modulating and/or treating existing levels of the constituents that form a signaling molecule for nucleotide activity comprising: measuring a level of phosphorus; measuring a level of magnesium; measuring a level of threonine; comparing the measured levels; and selecting and/or administering when necessary a treatment based upon the measured levels.

Another aspect of the invention is a method and kit for assessing, modulating and/or treating existing levels of the constituents that form a signaling molecule for nucleotide activity comprising: measuring a level of phosphorus; measuring a level of magnesium; measuring a level of serine; comparing the measured levels; and selecting and/or administering when necessary a treatment based upon the measured levels.

Another aspect of the invention is a method and kit for assessing, modulating and/or treating existing levels of the constituents that form the signaling molecule for nucleotide activity comprising: measuring a level of phosphorus; measuring a level of magnesium; measuring a level of histidine; comparing the measured levels; and selecting and/or administering when necessary a treatment based upon the measured levels.

Another aspect of the invention is a method and kit for assessing, modulating and/or treating existing levels of the constituents that form the signaling molecule for nucleotide activity comprising: measuring a level of phosphorus; measuring a level of magnesium; measuring a level of arginine; comparing the measured levels; and selecting and/or administering when necessary a treatment based upon the measured levels.

Another aspect of the invention is a method and kit for assessing, modulating and/or treating existing levels of the constituents that form the signaling molecule androstenone comprising: measuring a level of selenium; measuring a level of zinc; measuring a level of histidine; comparing the measured levels; and selecting and/or administering when necessary a treatment based upon the measured levels.

Another aspect of the invention is a method and kit for assessing, modulating and/or treating existing levels of the constituents that form the signaling molecule androstenediol comprising: measuring a level of selenium; measuring a level of zinc; measuring a level of arginine; comparing the measured levels; and selecting and/or administering when necessary a treatment based upon the measured levels.

Another aspect of the invention is a method and kit for assessing, modulating and/or treating existing levels of the constituents that form the signaling molecule androstenedione comprising: measuring a level of selenium; measuring a level of zinc; measuring a level of lysine; comparing the measured levels; and selecting and/or administering when necessary a treatment based upon the measured levels.

Another aspect of the invention is a method and kit for assessing, modulating and/or treating existing levels of the constituents that form the signaling molecule retinoblastoma-like protein (RBL) comprising: measuring a level of nickel; measuring a level of zinc; measuring a level of proline; comparing the measured levels; and selecting and/or administering when necessary a treatment based upon the measured levels.

Another aspect of the invention is a method and kit for assessing, modulating and/or treating existing levels of the signaling molecule retinoblastoma-like protein 1 (RBL1) comprising: measuring a level of copper; measuring a level of zinc; measuring a level of proline; comparing the measured levels; and selecting and/or administering when necessary a treatment based upon the measured levels.

Another aspect of the invention is a method and kit for assessing, modulating and/or treat existing levels of the signaling molecule retinoblastoma-like protein 2 (RBL2) comprising: measuring a level of iron; measuring a level of manganese; measuring a level of proline; comparing the measured levels; and selecting and/or administering when necessary a treatment based upon the measured levels.

Another aspect of the invention is a method and kit for assessing, modulating and/or treating existing levels of the signaling molecule Nerve Growth Factor (NGF) comprising: measuring a level of copper; measuring a level of zinc; measuring a level of glutamic acid; comparing the measured levels; and selecting and/or administering when necessary a treatment based upon the measured levels.

Another aspect of the invention is a method and kit for assessing, modulating and/or treating existing levels of the constituents that form the signaling molecule PUN comprising: measuring a level of copper; measuring a level of zinc; measuring a level of phenylalanine; comparing the measured levels; and selecting and/or administering when necessary a treatment based upon the measured levels.

Another aspect of the invention is a method and kit for assessing, modulating and/or treating existing levels of the constituents that form the signaling molecule ETS-1 comprising: measuring a level of copper; measuring a level of zinc; measuring a level of tyrosine; comparing the measured levels; and selecting and/or administering when necessary a treatment based upon the measured levels.

Another aspect of the invention is a method and kit for assessing, modulating and/or treating existing levels of the constituents that form the signaling molecule ETS-2 comprising: measuring a level of copper; measuring a level of zinc; measuring a level of tryptophan; comparing the measured levels; and selecting and/or administering when necessary a treatment based upon the measured levels.

Another aspect of the invention is a method and kit for assessing, modulating and/or treating existing levels of the constituents that form a signaling molecule for blood oxygenation comprising: measuring a level of sulfur; measuring a level of iron; measuring a level of serine; comparing the measured levels; and selecting and/or administering when necessary a treatment based upon the measured levels.

Another aspect of the invention is a method and kit for assessing, modulating and/or treating existing levels of the constituents that form a signaling molecule for blood oxygenation; measuring a level of sulfur; measuring a level of iron; measuring a level of threonine; and selecting and/or administering when necessary a treatment based upon the measured levels.

Another aspect of the invention is a method and kit for assessing, modulating and/or treat existing levels of the constituents that form a signaling molecule for blood oxygenation comprising; measuring a level of sulfur; measuring a level of iron; measuring a level of cysteine; and selecting and/or administering when necessary a treatment based upon the measured levels.

Another aspect of the invention is a method and kit for assessing, modulating and/or treating existing levels of the constituents that form a signaling molecule for the Huntingtin gene comprising: measuring a level of phosphorous; measuring a level of sulfur; measuring a level of histidine; comparing the measured levels; and selecting and/or administering when necessary a treatment based upon the measured levels.

Another aspect of the invention is a method and kit for assessing, modulating and/or treating existing levels of the constituents that form a signaling molecule for the Huntingtin gene comprising: measuring a level of phosphorous; measuring a level of sulfur; measuring a level of arginine; and selecting and/or administering when necessary a treatment based upon the measured levels.

Another aspect of the invention is a method and kit for assessing, modulating and/or treating existing levels of the constituents that form the signaling molecule lactoferrin comprising: measuring a level of iron; measuring a level of manganese; measuring a level of glutamic acid; comparing the measured levels; and selecting and/or administering when necessary a treatment based upon the measured levels.

Another aspect of the invention is a method and kit for assessing, modulating and/or treating existing levels of the constituents that the signaling molecule lactoferrin comprising: measuring a level of iron; measuring a level of manganese; measuring a level of alanine; comparing the measured levels; and selecting and/or administering when necessary a treatment based upon the measured levels.

Another aspect of the invention is a method and kit for assessing, modulating and/or treating existing levels of the constituents that form the signaling molecule lactoferrin comprising: measuring a level of iron; measuring a level of manganese; measuring a level of aspartic acid; comparing the measured levels; and selecting and/or administering when necessary a treatment based upon the measured levels.

Another aspect of the invention is a method and kit for assessing, modulating and/or treat existing levels of the constituents that form the signaling molecule hololactoferrin comprising: measuring a level of iron; measuring a level of manganese; measuring a level of phenylalanine; comparing the measured levels; and selecting and/or administering when necessary a treatment based upon the measured levels.

Another aspect of the invention is a method and kit for assessing, modulating and/or treating existing levels of the constituents that form the signaling molecule hololactoferrin comprising: measuring a level of iron in the individual; measuring a level of manganese in the individual; measuring a level of tyrosine in the individual; comparing the measured levels; and selecting and/or administering when necessary a treatment based upon the measured levels.

Another aspect of the invention is a method and kit for assessing, modulating and/or treating existing levels of the constituents that form the signaling molecule hololactoferrin comprising: measuring a level of iron in the individual; measuring a level of manganese in the individual; measuring a level of tryptophan in the individual; comparing the measured levels; and selecting and/or administering when necessary a treatment based upon the measured levels.

Another aspect of the invention is a method and kit for assessing, modulating and/or treating existing levels of the constituents that form the signaling molecule apolactoferrin comprising: measuring a level of iron; measuring a level of manganese; measuring a level of histidine; comparing the measured levels; and selecting and/or administering when necessary a treatment based upon the measured levels.

Another aspect of the invention is a method and kit for assessing, modulating and/or treating existing levels of the constituents that form the signaling molecule apolactoferrin comprising: measuring a level of iron; measuring a level of manganese; measuring a level of arginine; comparing the measured levels; and selecting and/or administering when necessary a treatment based upon the measured levels.

Another aspect of the invention is a method and kit for assessing, modulating and/or treating existing levels of the constituents that form the signaling molecule apolactoferrin comprising: measuring a level of iron; measuring a level of manganese; measuring a level of lysine; comparing the measured levels; and selecting and/or administering when necessary a treatment based upon the measured levels.

Another aspect of the invention is a method and kit for assessing, modulating and/or treating existing levels of the constituents that form the signaling molecule Interleukin 1(IL-1) comprising: measuring a level of aluminum; measuring a level of iron; measuring a level of nitric oxide; comparing the measured levels; and selecting and/or administering when necessary a treatment based upon the measured levels.

Another aspect of the invention is a method and kit for assessing, modulating and/or treating existing levels of the constituents that form the signaling molecule Interleukin 3 (IL-3) comprising: measuring a level of calcium; measuring a level of zinc; measuring a level of nitric oxide; comparing the measured levels; and selecting and/or administering when necessary a treatment based upon the measured levels.

Another aspect of the invention is a method and kit for assessing, modulating and/or treating existing levels of the constituents that form the signaling molecule Interleukin 5 (IL-5) comprising: measuring a level of selenium; measuring a level of zinc; measuring a level of nitric oxide; comparing the measured levels; and selecting and/or administering when necessary a treatment based upon the measured levels.

Another aspect of the invention is a method and kit for assessing, modulating and/or treating existing levels of the constituents that form the signaling molecule Interleukin 8 (IL-8) comprising: measuring a level of selenium; measuring a level of zinc; measuring a level of iodine; comparing the measured levels; and selecting and/or administering when necessary a treatment based upon the measured levels.

Another aspect of the invention is a method and kit for assessing, modulating and/or treating existing levels of the constituents that form the signaling molecule Interleukin 13 (IL-13) comprising: measuring a level of iron; measuring a level of manganese; measuring a level of nitric oxide; comparing the measured levels; and selecting and/or administering when necessary a treatment based upon the measured levels.

Another aspect of the invention is a method and kit for assessing, modulating and/or treating existing levels of the constituents that form the signaling molecule Interleukin 16 (IL-16) comprising: measuring a level of sulfur; measuring a level of iron; measuring a level of nitric oxide; comparing the measured levels; and selecting and/or administering when necessary a treatment based upon the measured levels.

Another aspect of the invention is a method and kit for assessing, modulating and/or treating existing levels of the constituents that form the signaling molecule Interleukin 18 (IL-18) comprising: measuring a level of copper; measuring a level of zinc; measuring a level of nitric oxide; comparing the measured levels; and selecting and/or administering when necessary a treatment based upon the measured levels.

Another aspect of the invention is a method and kit for assessing, modulating and/or treating existing levels of the constituents that form the signaling molecule Interleukin 23 (IL-23) comprising: measuring a level of bromine; measuring a level of iodine; measuring a level of nitric oxide; comparing the measured levels; and selecting and/or administering when necessary a treatment based upon the measured levels.

Another aspect of the invention is a method and kit for assessing, modulating and/or treating existing levels of the constituents that form the signaling molecule Interleukin 32 (IL-32) comprising: measuring a level of calcium; measuring a level of magnesium; measuring a level of nitric oxide; comparing the measured levels; and selecting and/or administering when necessary a treatment based upon the measured levels.

Another aspect of the invention is a method and kit for assessing, modulating and/or treating existing levels of the constituents that form the signaling molecule Interferon gamma (IFN-g) comprising: measuring a level of iron; measuring a level of manganese; measuring a level of hydrogen sulfide; comparing the measured levels; and selecting and/or administering when necessary a treatment based upon the measured levels.

Another aspect of the invention is a method and kit for assessing, modulating and/or treating existing levels of the constituents that form the signaling molecule retinoblastoma protein (pRB) comprising: measuring a level of copper; measuring a level of zinc; measuring a level of hydrogen sulfide; comparing the measured levels; and selecting and/or administering when necessary a treatment based upon the measured levels.

Another aspect of the invention is a method and kit for assessing, modulating and/or treating existing levels of the constituents that form the signaling molecule retinoblastoma protein 1 (pRB 1) comprising: measuring a level of nickel; measuring a level of zinc; measuring a level of hydrogen sulfide; comparing the measured levels; and selecting and/or administering when necessary a treatment based upon the measured levels.

Another aspect of the invention is a method and kit for assessing, modulating and/or treating existing levels of the constituents that form the signaling molecule troponin T comprising: measuring a level of calcium; measuring a level of zinc; measuring a level of phenylalanine; comparing the measured levels; and selecting and/or administering when necessary a treatment based upon the measured levels.

Another aspect of the invention is a method and kit for assessing, modulating and/or treating existing levels of the constituents that form the signaling molecule troponin C comprising; measuring a level of calcium; measuring a level of zinc; measuring a level of tyrosine; comparing the measured levels; and selecting and/or administering when necessary a treatment based upon the measured levels.

Another aspect of the invention is a method and kit for assessing, modulating and/or treating existing levels of the constituents that form the signaling molecule troponin I comprising: measuring a level of calcium; measuring a level of zinc; measuring a level of tryptophan; comparing the measured levels; and selecting and/or administering when necessary a treatment based upon the measured levels.

Another aspect of the invention is a method or kit for assessing, modulating and/or treating existing levels of the constituents that form the signaling molecules which are one of the 3 sub-units of Troponin TNI comprising: measuring a level of calcium; measuring a level of zinc; measuring a level of glutamic acid, alanine or aspartic acid; comparing the measured levels; and selecting and/or administering when necessary a treatment based upon the measured levels.

Another aspect of the invention is a method and kit for assessing, modulating and/or treating existing levels of the constituents that form the signaling molecule of one of the 3 sub-units of troponin TNT comprising: measuring a level of calcium; measuring a level of zinc; measuring a level of histidine; comparing the measured levels; and selecting and/or administering when necessary a treatment based upon the measured levels.

Another aspect of the invention is a method and kit for assessing, modulating and/or treating existing levels of the constituents that form the signaling molecule of one of the 3 sub-units of troponin TNT comprising: measuring a level of calcium; measuring a level of zinc; measuring a level of arginine; comparing the measured levels; and selecting and/or administering when necessary a treatment based upon the measured levels.

Another aspect of the invention is a method and kit for assessing, modulating and/or treating existing levels of the constituents that form the signaling molecule of one of the 3 sub-units of troponin TNT comprising: measuring a level of calcium; measuring a level of zinc; measuring a level of lysine; comparing the measured levels; and selecting and/or administering when necessary a treatment based upon the measured levels.

Another aspect of the invention is a method and kit for assessing, modulating and/or treating existing levels of the constituents that form the signaling molecule troponin TNC comprising: measuring a level of calcium; measuring a level of zinc; measuring a level of leucine; comparing the measured levels; and selecting and/or administering when necessary a treatment based upon the measured levels.

Another aspect of the invention is a method and kit for assessing, modulating and/or treating existing levels of the constituents that form the signaling molecule troponin TNC comprising: measuring a level of calcium; measuring a level of zinc; measuring a level of isoleucine; comparing the measured levels; and selecting and/or administering when necessary a treatment based upon the measured levels.

Another aspect of the invention is a method and kit for assessing, modulating and/or treating existing levels of the constituents that form the signaling molecule troponin TNC comprising: measuring a level of calcium; measuring a level of zinc; measuring a level of valine; and selecting and/or administering when necessary a treatment based upon the measured levels.

Another aspect of the invention is a method and kit for assessing, modulating and/or treating existing levels of the constituents that form the signaling molecule Lefty 1 comprising: measuring a level of calcium; measuring a level of zinc; measuring a level of serine; and selecting and/or administering when necessary a treatment based upon the measured levels.

Another aspect of the invention is a method and kit for assessing, modulating and/or treating existing levels of the constituents that form the signaling molecule Lefty 2 comprising; measuring a level of calcium; measuring a level of zinc; measuring a level of cysteine; and selecting and/or administering when necessary a treatment based upon the measured levels.

Another aspect of the invention is a method and kit for assessing, modulating and/or treating existing levels of the constituents that form the signaling molecule Nodal comprising; measuring a level of calcium; measuring a level of zinc; measuring a level of threonine; and selecting and/or administering when necessary a treatment based upon the measured levels.

Another aspect of the invention is a method and kit for assessing, modulating and/or treating existing levels of the constituents that form the signaling molecule SynCAM2 comprising; measuring a level of calcium; measuring a level of magnesium; measuring a level of threonine; and selecting and/or administering when necessary a treatment based upon the measured levels.

Another aspect of the invention is a method and kit for assessing, modulating and/or treating existing levels of the constituents that form the signaling molecule SynCAM1 comprising; measuring a level of calcium; measuring a level of magnesium; measuring a level of serine; and selecting and/or administering when necessary a treatment based upon the measured levels.

Another aspect of the invention is a method and kit for assessing, modulating and/or treating existing levels of the constituents that form the signaling molecule SynCAM3 comprising; measuring a level of calcium; measuring a level of magnesium; measuring a level of cysteine; and selecting and/or administering when necessary a treatment based upon the measured levels.

Another aspect of the invention is a method and kit for assessing, modulating and/or treating existing levels of the constituents that form the signaling molecule T4 thyroid hormone comprising; measuring a level of bromine; measuring a level of iodine; measuring a level of serine; and selecting and/or administering when necessary a treatment based upon the measured levels.

Another aspect of the invention is a method and kit for assessing, modulating and/or treating existing levels of the constituents that form the signaling molecule T3 thyroid hormone comprising; measuring a level of bromine; measuring a level of iodine; measuring a level of cysteine; and selecting and/or administering when necessary a treatment based upon the measured levels.

Another aspect of the invention is a method and kit for assessing, modulating and/or treating existing levels of the constituents that form the signaling molecule T2 thyroid hormone comprising: measuring a level of bromine; measuring a level of iodine; measuring a level of threonine; comparing the measured levels; and selecting and/or administering when necessary a treatment based upon the measured levels.

Another aspect of the invention is a method and kit for assessing, modulating and/or treating existing levels of the constituents that form the signaling molecule secretin comprising: measuring a level of bromine; measuring a level of iodine; measuring a level of glutamine acid; comparing the measured levels; and selecting and/or administering when necessary a treatment based upon the measured levels.

Another aspect of the invention is a method and kit for assessing, modulating and/or treating existing levels of the constituents that form the signaling molecule gastric inhibitory peptide (GIP) comprising: measuring a level of bromine; measuring a level of iodine; measuring a level of aspartic acid; comparing the measured levels; and selecting and/or administering when necessary a treatment based upon the measured levels.

Another aspect of the invention is a method and kit for assessing, modulating and/or treating existing levels of the constituents that form the signaling molecule vasoactive intestinal peptide (VIP) comprising: measuring a level of bromine in the individual; measuring a level of iodine; measuring a level of alanine; comparing the measured levels; and selecting and/or administering when necessary a treatment based upon the measured levels.

Another aspect of the invention is a method and kit for assessing, modulating and/or treating existing levels of the constituents that forms the signaling molecule alpha aminobutyric acid (AABA) comprising: measuring a level of iron; measuring a level of sulfur; measuring a level of glutamic acid; comparing the measured levels; and selecting and/or administering when necessary a treatment based upon the measured levels.

Another aspect of the invention is a method and kit for assessing, modulating and/or treating existing levels of the constituents that form the signaling molecule beta aminobutyric acid (BABA) comprising: measuring a level of iron; measuring a level of sulfur; measuring a level of aspartic acid; comparing the measured levels; and selecting and/or administering when necessary a treatment based upon the measured levels.

Another aspect of the invention is a method and kit for assessing, modulating and/or treating existing levels of the constituents that form the signaling molecule gamma aminobutyric acid (GABA) comprising: measuring a level of iron; measuring a level of sulfur; measuring a level of alanine; comparing the measured levels; and selecting and/or administering when necessary a treatment based upon the measured levels.

Another aspect of the invention is a method and kit for assessing, modulating and/or treating existing levels of the constituents that form the signaling molecule Nuclear Respiratory Factor 1 (NRF-1) comprising: measuring a level of selenium; measuring a level of zinc; measuring a level of leucine; comparing the measured levels; and selecting and/or administering when necessary a treatment based upon the measured levels.

Another aspect of the invention is a method and kit for assessing, modulating and/or treating existing levels of the constituents that form the signaling molecule Nuclear Respiratory Factor 2 (NRF-2) comprising: measuring a level of selenium; measuring a level of zinc; measuring a level of isoleucine; comparing the measured levels; and selecting and/or administering when necessary a treatment based upon the measured levels.

Another aspect of the invention is a method and kit for assessing, modulating and/or treating existing levels of the constituents that form the signaling molecule Nuclear Respiratory Factor 3 (NRF-3) comprising: measuring a level of selenium; measuring a level of zinc; measuring a level of valine; comparing the measured levels; and selecting and/or administering when necessary a treatment based upon the measured levels.

Another aspect of the invention is a method and kit for assessing, modulating and/or treating existing levels of the constituents that form the signaling molecule NPY-PYY-PP comprising: measuring a level of Interleukin 12; measuring a level of Interleukin 18; measuring a level of Interleukin 16; comparing the measured levels; and selecting and/or administering when necessary a treatment based upon the measured levels.

Another aspect of the invention is a method and kit for assessing, modulating and/or treating existing levels of the constituents that form the signaling molecule Caspase 1 comprising: measuring a level of iron; measuring a level of manganese; measuring a level of threonine; comparing the measured levels; and selecting and/or administering when necessary a treatment based upon the measured levels.

Another aspect of the invention is a method and kit for assessing, modulating and/or treating existing levels of the constituents that form the signaling molecule Caspase 2 comprising: measuring a level of iron; measuring a level of manganese; measuring a level of serine; comparing the measured levels; and selecting and/or administering when necessary a treatment based upon the measured levels.

Another aspect of the invention is a method and kit for assessing, modulating and/or treating existing levels of the constituents that form the signaling molecule Caspase 3 comprising: measuring a level of iron; measuring a level of manganese; measuring a level of cysteine; comparing the measured levels; and selecting and/or administering when necessary a treatment based upon the measured levels.

Another aspect of the invention is a method and kit for assessing, modulating and/or treating existing levels of the constituents that form the signaling molecule nitrogenase comprising: measuring a level of iron; measuring a level of sulfur; measuring a level of molybdenum; comparing the measured levels; and selecting and/or administering when necessary a treatment based upon the measured levels.

Another aspect of the invention is a method and kit for assessing, modulating and/or treating existing levels of the constituents that form the signaling molecule DISC 1 comprising: measuring a level of sodium; measuring a level of potassium; measuring a level of proline; comparing the measured levels; and selecting and/or administering when necessary a treatment based upon the measured levels.

Another aspect of the invention is a method and kit for assessing, modulating and/or treating existing levels of the constituents that form the signaling molecule DISC 2 comprising: measuring a level of sodium; measuring a level of potassium; measuring a level of glutamine; comparing the measured levels; and selecting and/or administering when necessary a treatment based upon the measured levels.

Another aspect of the invention is a method and kit for assessing, modulating and/or treating existing levels of the constituents that form the signaling molecule TSNAX comprising: measuring a level of sodium; measuring a level of potassium; measuring a level of glycine; and selecting and/or administering when necessary a treatment based upon the measured levels.

Another aspect of the invention is a method and kit for assessing, modulating and/or treating existing levels of the constituents that form the signaling molecule lipoprotein receptor related protein 4 (LRP-4) comprising: measuring a level of sodium; measuring a level of potassium; measuring a level of serine; and selecting and/or administering when necessary a treatment based upon the measured levels.

Another aspect of the invention is a method and kit for assessing, modulating and/or treating existing levels of the constituents that form the signaling molecule lipoprotein receptor related protein 5 (LRP-5) comprising: measuring a level of sodium; measuring a level of potassium; measuring a level of threonine; and selecting and/or administering when necessary a treatment based upon the measured levels.

Another aspect of the invention is a method and kit for assessing, modulating and/or treating existing levels of the constituents that form the signaling molecule lipoprotein receptor related protein 6 (LRP-6) comprising: measuring a level of sodium; measuring a level of potassium; measuring a level of cysteine; and selecting and/or administering when necessary a treatment based upon the measured levels.

Another aspect of the invention is a method and kit for assessing, modulating and/or treating existing levels of constituents that form the signaling molecule ceruloplasmin comprising: measuring a level of copper; measuring a level of zinc; measuring a level of iron; and selecting and/or administering when necessary a treatment based upon the measured levels.

Another aspect of the invention is a method and kit for assessing, modulating and/or treating existing levels of constituents that form the signaling molecule Interleukin 2 (IL-2) comprising: measuring a level of sodium; measuring a level of potassium; measuring a level of chloride; and selecting and/or administering when necessary a treatment based upon the measured levels.

Another aspect of the invention is a method and kit for assessing, modulating and/or treating existing levels of constituents that form the signaling molecule Interleukin 11 (IL-11) comprising: measuring a level of phosphorus; measuring a level of calcium; measuring a level of magnesium; and selecting and/or administering when necessary a treatment based upon the measured levels.

Another aspect of the invention is a method and kit for assessing, modulating and/or treating existing levels of constituents that form the signaling molecule Interleukin 4 (IL-4) comprising: measuring a level of nickel; measuring a level of zinc; measuring a level of boron; and selecting and/or administering when necessary a treatment based upon the measured levels.

Another aspect of the invention a method and kit for assessing, modulating and/or treating existing levels of constituents that form the signaling molecule Interleukin 12 (IL-12) comprising: measuring a level of nickel; measuring a level of zinc; measuring a level of nitric oxide; and selecting and/or administering when necessary a treatment based upon the measured levels.

Another aspect of the invention a method and kit for assessing, modulating and/or treating existing levels of constituents that form the signaling molecule Interleukin 33 (IL-33) comprising: measuring a level of sodium; measuring a level of potassium; measuring a level of nitric oxide; and selecting and/or administering when necessary a treatment based upon the measured levels.

Another aspect of the invention is a method and kit for assessing, modulating and/or treating the homeostatic relationships between the levels of the neurohormones aldosterone, oxytocin, serotonin, adrenaline and cortisol comprising: measuring the levels of these neurohormones; comparing the measured levels; and selecting and/or administering when necessary a treatment based upon the measured levels.

Still another aspect of the invention is a method and kit for assessing, modulating and/or treating the homeostatic relationships between the levels of the neurohormones norepinephrine, prolactin and dopamine comprising: measuring the levels of these neurohormones; comparing the measured levels; and selecting and/or administering when necessary a treatment based upon the measured levels.

### BRIEF DESCRIPTION OF THE FIGURES

FIG. 1 shows the relationship between constituents which form the signaling molecule tight junction protein 1.

FIG. 2 is a flow chart of the method for establishing a treatment for restoring homeostasis based upon the relationship of the substances shown in FIG. 1.

FIG. 3 is a schematic diagram showing the system for measuring the levels of substances from within an individual or animal.

FIG. 4 shows the method of using the system for measuring the levels of substances within an individual or animal, in accordance with an embodiment of the present invention.

### DETAILED DESCRIPTION OF THE INVENTION

Associated substances create the homeostasis essential to maintain life for humans as well as animals. These substances can modulate each other in various ways.

Several terms are used herein and are defined as follows:

The term “modulator” means an agent(s), substance(s) or mechanism(s) that facilitates the maintenance of homeostasis between associated substances. “Associated substances” may be hormones, elements listed on the Periodic Chart, proteins, amino acids, bacteria, virus and gases. When associated substances are “modulated” they can decrease intensity of stimulatory processes or substances, or increase intensity of compensatory ones. When a substance is indicated as being a “modulator” it means that it plays a role along with the other associated substances in establishing a homeostatic balance. It should be understood that while one substance may be indicated as the modulator, it is the interaction between all three substances that affects the homeostatic levels.

The term “catalyst” means the driver, which initiates modulation to take place. Such catalysts can include signaling molecules, hormones, protein kinases, gases and countless other signaling mechanisms.

The term “Homeostatic Relationship (Homeostasis)” means the balance or equilibrium between substances. The relationship does not have to be 50%-50% but can vary depending upon the substances in question.

The term “Extracellular Matter (ECM)” means all body fluid outside of cells and blood; including but not limited to cerebrospinal fluid, lymph, chyle, interstitial fluid, saliva, tears, transcellular fluid, amniotic fluid, sperm, and sputum. These substances will also include breath as a source for testing.

The term “Mental Functioning Activity (MFA)” means the rate of activity attributable to the neural firing rate in the brain and elsewhere in the body.

The invention addresses the diagnostic analysis of the corollary relationships between substances that provide and maintain homeostasis in an individual and affect bodily processes.

The data for measuring homeostasis between substances and/or with associated substances can vary from one individual or specimen to another due to environmental factors, basic metabolism, genetic make-up and other factors. Data derived from the application of the invention will allow for diagnostic analysis and control of levels of the substances in order to prevent their depletion, to maintain adequate levels to prevent disruption as well as to create recommendations for which substances may counteract with others and the levels of which substances must be modified on an ongoing basis in order to provide optimal health/well being through homeostasis.

Since signaling molecules are essential to create and maintain defenses for the organs, glands and operating systems of the body, as well as to provide the energy to sustain life. Establishing corollary relationships between the substances or constituents necessary for the production of signaling molecules is critical. Tests for the corollary relationship between the substances that form the signaling molecules will enable analysis to prevent depletion of the body's defense in order to prevent illness as well as to increase levels of nutritional energy to strengthen the body to offset the effects of existing ailments and diseases.

Some examples of substances that operate as first, second and third modulators within an individual in the formation of signaling molecules include, but are not limited to, calcium and magnesium in conjunction with phenylalanine having the role/process in/for the formation of the associated substance Tight Junction Protein 1 (TJP-1); calcium and magnesium in conjunction with tyrosine having the role/process in/for the formation of Tight Junction Protein 2 (TJP-2); calcium and magnesium in conjunction with tryptophan having the role/process in/for the formation of Tight Junction Protein 3 (TJP-3); iron and sulfur in conjunction with nitric oxide having the role/process in/for the formation of aldosterone; iron and sulfur in conjunction with arginine having the role/process in/for the formation of oxytocin; iron and sulfur in conjunction with histidine having the role/process in/for the formation of serotonin; zinc and nickel in conjunction with tryptophan having the role/process in/for the formation of adrenaline; and zinc and nickel in conjunction with nitric oxide having the role/process in/for the formation of cortisol.

Additional examples include, but are not limited to, iron and sulfur in conjunction with phosphorus having the role/process in/for the formation of p53; copper and zinc in conjunction with calcium having the role/process in/for the formation of p63; nickel and zinc in conjunction with boron having the role/process in/for the formation of p73; phosphorus and calcium in conjunction with leucine having the role/process in/for the formation of vitamin K2; phosphorus and calcium in conjunction with valine having the role/process in/for the formation of vitamin K3 (a.k.a. MK4); phosphorus and calcium in conjunction with nitric oxide having the role/process in/for the formation of Interleukin 15; and phosphorus and chromium in conjunction with nitric oxide having the role/process in/for the formation of Interleukin 17.

Additional examples include, but are not limited to, bromine and iodine in conjunction with phenylalanine having the role/process in/for the formation of nitric oxide synthase 3; bromine and iodine in conjunction with tyrosine having the role/process in/for the formation of nitric oxide synthase 3; bromine and iodine in conjunction with tryptophan having the role/process in/for the formation of nitric oxide synthase 3; iron and sulfur in conjunction with threonine having the role/process in/for the formation of HMOX-1; iron and sulfur in conjunction with serine having the role/process in/for the formation of HMOX-2; and iron and sulfur in conjunction with cysteine having the role/process in/for the formation of HMOX-3

Additional examples include, but are not limited to, phosphorus and calcium in conjunction with phenylalanine having the role/process in/for the formation of TIG-1; phosphorus and calcium in conjunction with tyrosine having the role/process in/for the formation of TIG-2; phosphorus and calcium in conjunction with tryptophan having the role/process in/for the formation of TIG-3; phosphorus and chromium in conjunction with tyrosine having the role/process in/for the formation of HER-1; phosphorus and chromium in conjunction with phenylalanine having the role/process in/for the formation of HER-2; phosphorus and chromium in conjunction with tryptophan having the role/process in/for the formation of HER-3; phosphorus and calcium in conjunction with histidine having the role/process in/for the formation of acetylcholinesterase; phosphorus and calcium in conjunction with arginine having the role/process in/for the formation of butylcholinesterase; phosphorus and sulfur in conjunction with histidine having the role/process in/for the formation of the signaling molecule for nucleotide activity; phosphorus and sulfur in conjunction with arginine having the role/process in/for the formation of the signaling molecule for nucleotide activity; and phosphorous and silicon in conjunction with histidine having the role/process in/for the formation of a signaling molecule for nucleotide activity.

Additional examples include, but are not limited to, phosphorus and silicon in conjunction with arginine having the role/process in/for the formation of a signaling molecule for nucleotide activity; phosphorus and silicon in conjunction with lysine having the role/process in/for formation of a signaling molecule for nucleotide activity; phosphorus and silicon in conjunction with threonine having the role/process in/for the formation of a signaling molecule for nucleotide activity; phosphorus and silicon in conjunction with serine having the role/process in/for the formation of a signaling molecule for nucleotide activity; phosphorus and magnesium in conjunction with threonine having the role/process in/for the formation of a signaling molecule for nucleotide activity; phosphorous and magnesium in conjunction with serine having the role/process in/for formation of a signaling molecule for nucleotide activity; phosphorus and magnesium in conjunction with histidine having the role/process in/for the formation of a signaling molecule for nucleotide activity; and phosphorus and magnesium in conjunction with arginine having the role/process in/for the formation of a signaling molecule for nucleotide activity.

Additional examples include, but are not limited to, selenium and zinc in conjunction with histidine having the role/process in/for the formation of a signaling molecule for androstenone; selenium and zinc in conjunction with arginine having the role/process in/for the formation of a signaling molecule for androstenediol; selenium and zinc in conjunction with lysine having the role/process in/for the formation of a signaling molecule for androstenedione; nickel and zinc in conjunction with proline having the role/process in/for the formation of retinoblastoma-like protein (RBL); copper and zinc in conjunction with proline having the role/process in/for the formation of retinoblastoma-like protein 1 (RBL-1); iron and manganese in conjunction with proline having the role/process in/for the formation of retinoblastoma-like protein 2 (RBL-2); and copper and zinc in conjunction with glutamine acid having the role/process in/for the formation of Nerve Growth Factor (NGF).

Additional examples include, but are not limited to, copper and zinc in conjunction with phenylalanine having the role/process in/for the formation of PTEN; copper and zinc in conjunction with tyrosine having the role/process in/for the formation of ETS-1; copper and zinc in conjunction with tryptophan having the role/process in/for the formation of ETS-2; sulfur and iron in conjunction with serine having the role/process in/for the formation of one of the signaling molecules for blood oxygenation; sulfur and iron in conjunction with threonine having the role/process in/for the formation one of the signaling molecules for blood oxygenation; and sulfur and iron in conjunction with cysteine having the role/process in/for the formation of a signaling molecule for blood oxygenation.

Additional examples include, but are not limited to, phosphorus and sulfur in conjunction with histidine having the role/process in/for the formation of a signaling molecule for the Huntingtin gene; phosphorus and sulfur in conjunction with arginine having the role/process in/for the formation of a signaling molecule for the Huntingtin gene; iron and manganese in conjunction with glutamic acid having the role/process in/for the formation of one of the forms of lactoferrin; iron and manganese in conjunction with alanine having the role/process in/for the formation of another form of lactoferrin; iron and manganese in conjunction with aspartic acid having the role/process in/for the formation of another form of lactoferrin; iron and manganese in conjunction with phenylalanine having the role/process in/for the formation of one of the forms of hololactoferrin; iron and manganese in conjunction with tyrosine having the role/process in/for the formation of another form of hololactoferrin; iron and manganese in conjunction with tryptophan having the role/process in/for the formation of another form of hololactoferrin; iron and manganese in conjunction with histidine having the role/process in/for the formation of one of the forms of apolactoferrin; iron and manganese in conjunction with arginine having the role/process in/for the formation of one of the forms of apolactoferrin; and iron and manganese in conjunction with lysine having the role/process in/for the formation of one of the forms of apolactoferrin.

Additional examples include, but are not limited to, aluminum and iron in conjunction with nitric oxide having the role/process in/for the formation of Interleukin 1 (IL-1); calcium and zinc in conjunction with nitric oxide having the role/process in/for the formation of Interleukin 3 (IL-3); selenium and zinc in conjunction with nitric oxide having the role/process in/for the formation of Interleukin 5 (IL-5); selenium and zinc in conjunction with iodine having the role/process in/for the formation of Interleukin 8 (IL-8); iron and manganese in conjunction with nitric oxide having the role/process in/for the formation of Interleukin 13 (IL-13); sulfur and iron in conjunction with nitric oxide having the role/process in/for the formation of Interleukin 16 (IL-16); copper and zinc in conjunction with nitric oxide having the role/process in/for the formation of Interleukin 18 (IL-18); bromine and iodine in conjunction with nitric oxide having the role/process in/for the formation of Interleukin 23 (IL-23); and calcium and magnesium in conjunction with nitric oxide having the role/process in/for the formation of Interleukin 32 (IL-32).

Additional examples include, but are not limited to, iron and manganese in conjunction with hydrogen sulfide having the role/process in/for the formation of Interferon-gamma (IFN-g); copper and zinc in conjunction with hydrogen sulfide having the role/process in/for the formation of retinoblastoma protein (pRB); and nickel and zinc in conjunction with hydrogen sulfide having the role/process in/for the formation of retinoblastoma protein 1 (pRB-1).

Additional examples include, but are not limited to, calcium and zinc in conjunction with phenylalanine having the role/process in/for the formation of Troponin T; calcium and zinc in conjunction with tyrosine having the role/process in/for the formation of Troponin C; calcium and zinc in conjunction with tryptophan having the role/process in/for the formation of Troponin I; calcium and zinc in conjunction with histidine having the role/process in/for the formation of one of the 3 sub-units of Troponin TNT; calcium and zinc in conjunction with arginine having the role/process in/for the formation of one of the 3 sub-units of Troponin TNT; calcium and zinc in conjunction with lysine having the role/process in/for the formation of the 3 sub-units of Troponin TNT; calcium and zinc in conjunction with leucine having the role/process in/for the formation of the 3 sub-units of Troponin TNC; calcium and zinc in conjunction with isoleucine having the role/process in/for the formation of the 3 sub-units of Troponin TNC; and calcium and zinc in conjunction with valine having the role/process in/for the formation of the 3 sub-units of Troponin TNC.

Additional examples include, but are not limited to, calcium and zinc acid in conjunction with serine having the role/process in/for the formation of the signaling molecule Lefty 1; calcium and zinc in conjunction with cysteine having the role/process in/for the formation of the signaling molecule Lefty 2; calcium and zinc in conjunction with threonine having the role/process in/for the formation of the signaling molecule NODAL; calcium and magnesium in conjunction with threonine having the role/process in/for the formation of the signaling molecule SynCAM2; calcium and magnesium in conjunction with serine having the role/process in/for the formation of the signaling molecule SynCAM1; and calcium and magnesium in conjunction with cysteine having the role/process in/for the formation of the signaling molecule SynCAM3.

Additional examples include, but are not limited to, bromine and iodine in conjunction with serine having the role/process in/for the formation of T4 thyroid hormone; bromine and iodine in conjunction with cysteine having the role/process in/for the formation of T3 thyroid hormone; bromine and iodine in conjunction with threonine having the role/process in/for the formation of T2 thyroid hormone; bromine and iodine in conjunction with glutamic acid having the role/process in the formation of secretin; bromine and iodine in conjunction with aspartic acid having the role/process in/for the formation of gastric inhibitory peptide (GIP); bromine and iodine in conjunction with alanine having the role/process in the formation of vasoactive intestinal peptide (VIP); iron and sulfur in conjunction with glutamic acid having the role/process in/for the formation of alpha aminobutyric acid; iron and sulfur in conjunction with aspartic acid having the role/process in/for the formation of beta aminobutyric acid; and iron and sulfur in conjunction with alanine having the role/process in/for the formation of gamma aminobutyric acid.

Additional examples include, but are not limited to, selenium and zinc in conjunction with leucine having the role/process in/for the formation of Nuclear Respiratory Factor 1 (NRF-1); selenium and zinc in conjunction with isoleucine having the role/process in/for the formation of Nuclear Respiratory Factor 2 (NRF-2); selenium and zinc in conjunction with valine having the role/process in/for the formation of Nuclear Respiratory Factor 3 (NRF-3); and Interleukin 12 and Interleukin 16 in conjunction with Interleukin 18 having the role/process in/for the formation or neuropeptide Y (NPY)-polypeptide y (PYY) and pancreatic peptide (PP).

Additional examples include, but are not limited to, iron and manganese in conjunction with threonine having the role/process in/for the formation of Caspase 1; iron and manganese in conjunction with serine having the role/process in/for the formation of Caspase 2; iron and manganese in conjunction with cysteine having the role/process in/for the formation of Caspase 3; and iron and sulfur in conjunction with molybdenum having the role/process in/for the formation of nitrogenase.

Additional examples include, but are not limited to, potassium and sodium in conjunction with proline having the role/process in the formation of DISC1; sodium and potassium in conjunction with glutamine having the role/process in/for the formation of DISC2; sodium and potassium in conjunction with glycine having the role/process in/for the formation of TSNAX; sodium and potassium in conjunction with serine having the role/process in/for the formation of lipoprotein receptor related protein 4 (LRP-4); sodium and potassium in conjunction with threonine having the role/process in/for the formation of lipoprotein receptor related protein 5 (LRP-5); sodium and potassium in conjunction with cysteine having the role/process in/for the formation of lipoprotein receptor related protein 6 (LRP-6); and copper and zinc in conjunction with iron having the role/process in/for the formation of ceruloplasmin.

Additional examples include, but are not limited to, sodium and potassium in conjunction with chlorine having the role/process in/for the formation of Interleukin 2; phosphorus and calcium in conjunction with magnesium having the role/process in/for the formation of Interleukin 11; nickel and zinc in conjunction with boron having the role/process in/for the formation of Interleukin 4; nickel and zinc in conjunction with nitric oxide having the role/process in/for the formation of Interleukin 12; and sodium and potassium in conjunction with nitric oxide having the role/process in/for the formation of Interleukin 33 (IL-33).

Additional examples include, but are not limited to, calcium and zinc in conjunction with glutamic acid having the role/process in/for the formation of one of the 3 sub-units of Troponin TNI; calcium and zinc in conjunction with alanine having the role/process in/for the formation of one of the 3 sub-units of Troponin TNI; and calcium and zinc in conjunction with aspartic acid having the role/process in/for the formation of the 3 sub-units of Troponin TNI.

Additional examples include, but are not limited to, the correlation of the levels of the neurohormes aldosterone, oxytocin, serotonin, adrenaline and cortisol to assess their homeostatic relationships and the correlation of the levels of the neurohormes norepinephrine, prolactin and dopamine to assess their homeostatic relationships.

It should be noted that some substances can function as part of a group of associated substances and also be a modulator for another group of associated substances. Additionally, it should be understood that while specific processes and roles are noted above as being affected by the associated substances, other processes and roles may also be affected. It should also be noted that while an example is discussed below, it is exemplary of the process that may be obtained with all listed associated substances herein.

Correlation between base values expressed when the range of homeostasis is determined and individual test results can be used as a means of assessing levels or disruptions between associated substances or the existence of increases in the levels of substances in order to maintain homeostasis. Methods of diagnostic analysis may also detect the presence of catalysts as markers for the existence of a disruption within an associated substance that the body is in the process of correcting. Detection of such catalysts may be included as a factor in the process of analyzing the correlation of substances within associated substances.

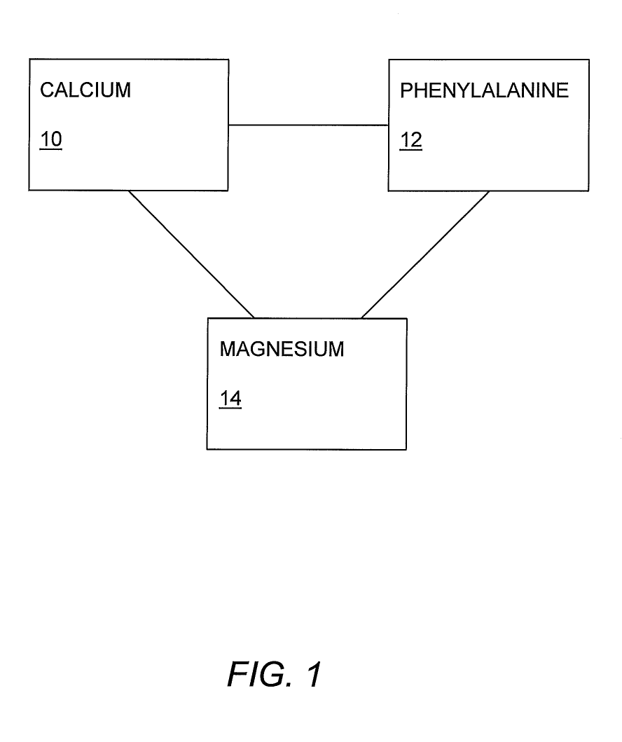

Described herein is an example of the determination of homeostatic levels of one embodiment of the sets of associated substances disclosed herein. In this embodiment, the relationship of which is depicted in FIG. 1, the first set of modulators is the pair calcium 10 and phenylalanine 12, which forms a homeostatic relationship with magnesium 14. This relationship plays a role in the formation of the signaling molecule tight junction protein 1 (TJP-1) and the homeostatic establishment of it.

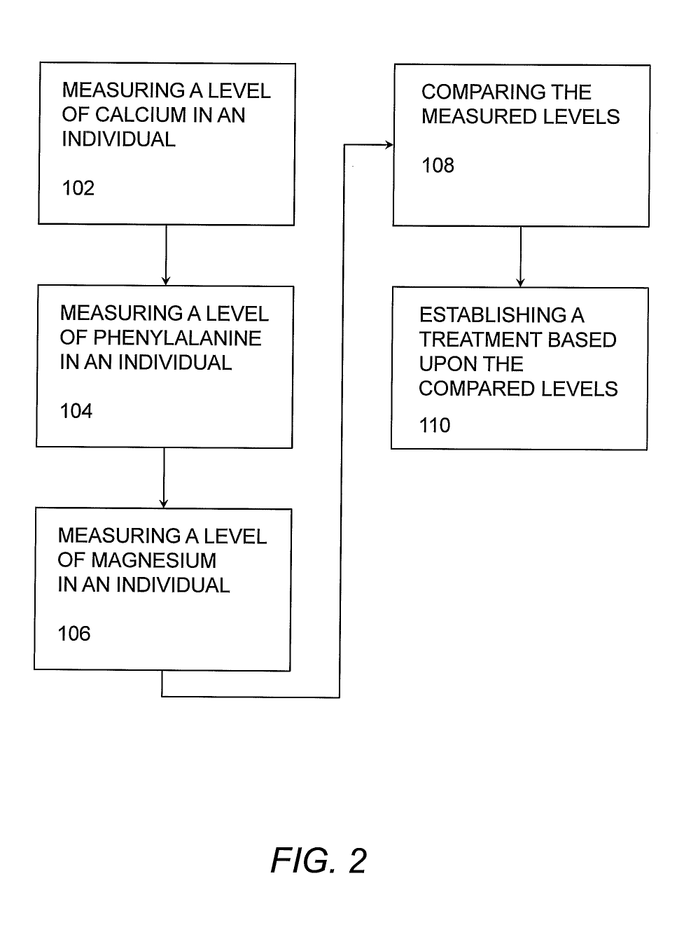

FIG. 2 shows the method of evaluating the homeostatic levels of the first modulator, calcium 10, and the second modulator, phenylalanine 12, in order to provide a base index for administering medication and performing analysis of an individual. In step 102, the level of calcium 10 is measured in an individual. The level of calcium 10 may be measured by a variety of diagnostic tests that include, but are not limited to the analysis of diagnostic imaging, assessment of electric activity in the body by EEG and EKG-like devices that indicates activity and the analysis of various fluids and excretions within and produced by the body as well as reflectance pulse oximetry or transmission pulse oximetry-like analysis using skin or other parts of the body. Examples of these fluids and excretions include, but are not limited to: blood and its components; serum; urine; fecal matter; collagen; chyle; interstitial fluid (tissue fluid); lymph; extracellular fluid; amniotic fluid; sweat tears; saliva; mucus; phlegm; hair; fingernails; bone marrow and breath.

In addition to the conventional diagnostic analysis of levels of substances from the aforementioned sources, various existing and yet to be developed diagnostic processes that measure outcomes resulting from these substances can be utilized. These devices may include, but are not limited to, EEG, PET scans, use of MEG machines, SPECT analysis, functional and diffusion MRI technologies as well as other iterations thereof (fMRI and dMRI respectively), CT scans, ultrasound and breath analysis devices, as well as reflectance pulse oximetry or transmission pulse oximetery-like analysis using skin or other parts of the body.

Correlation between base values expressed when the range of homeostasis is determined and individual test results may be used as a means of assessing levels or disruptions between associated substances or the existence of increases in the levels of modulators in order to maintain homeostasis. Methods of diagnostic analysis may also detect the presence of catalysts as markers for the existence of a disruption within a modulated associated substance that the body is in the process of remediating. Detection of such catalysts is included as a factor in the process of analyzing the correlation of substances within modulated associated substances.

This invention provides the biological foundation that will enable monitoring relationships within modulated associated substances of substances as they relate to medical care; including wellness, prevention and treatment pertaining to the mind (brain) and body.

The measured level of calcium 10 in the body or environment may be expressed in various units depending upon the test employed. This level can be expressed by the variable X. There is a preferred range in which X may fall that varies depending upon the type of test that is used in order to measure the level of calcium 10. In an embodiment of the present invention a standard type of test is used, such as a blood test.

In step 104, the level of the second modulator, phenylalanine 12, in an individual is measured. This level can be measured using one of the various processes listed above. The measured level of phenylalanine 12 is preferably expressed in the same units of measurement as that used in the measurement of calcium 10 so as to more readily compare the levels with respect to each other in order to obtain a base level. This level can be expressed by the variable Y. There is a preferred range in which Y may fall that varies depending upon the type of test that is used. In an embodiment of the present invention a standard type of test is used, such as a blood test.

In step 106, the level of the third modulator, magnesium 14, in an individual is measured. This level can be measured using one of the various processes listed above. The measured level of magnesium 14 is preferably expressed in the same units of measurement as that used in the measurement of calcium 10 and/or phenylalanine 12 so as to more readily compare the levels with respect to each other in order to obtain a base level. This level can be expressed by the variable Z. There is a preferred range in which Z may fall that varies depending upon the type of test that is used. In an embodiment of the present invention a standard type of test is used, such as blood test.

In step 108, the measured levels X, Y and Z are then compared to standard levels. This may be done in a number of different ways. One possible way in which this can be accomplished is to total the values of all of the levels (X+Y+Z) in order to establish sum level C. It is also possible to establish acceptable ratios for the values X:Y, X:Z and Y:Z. The sum level C can be used in step 110 in order to establish a treatment or diagnosis of the individual that can be used in determining the provision of medication and other matters related to the measured levels. In particular the levels may be used to determine if more magnesium 14 may need to be supplied in order to place the level of phenylalanine 12 or calcium 10 back to proper levels and to promote the formation of the signaling molecule, Tight Junction Protein 1 (TJP-1), and establish homeostasis.

A nonlimiting example is provided below using hypothetical numbers so as to facilitate understanding of this embodiment of the invention.

In the process a level of calcium 10 is determined from analyzing a blood test. Measuring calcium 10 via a blood test results in a value for X of 6 in the appropriate units. Acceptable ranges for X may be between 5-10. Measuring phenylalanine 12 via a blood test results in a number for Y of 5, in the appropriate units, from within a range of 1-10. Measuring magnesium 14 via the blood test results in a number for Z of 15, in the appropriate units, from within an acceptable range of between 1-10. These numbers are totaled and result in a C value of 26. A value for C between 10 and 20 may indicate a normal level.

A value for C that falls below 10 or above 20 may signal the need to provide a treatment that administers additional calcium 10, phenylalanine 12, or magnesium 14 to the individual. Alternatively, it may signal that the diet should be altered in order to reduce the levels of these substances. In this instance, levels of calcium 10 may be reduced. Alternatively, if the level for Z was 5, and the levels for X and Y remained the same, pills containing calcium 10 may be administered in order to increase levels.

Monitoring of the individual may be performed periodically until the levels return to accepted ranges. Various means of administration are contemplated herein in addition to pill form, such as intravenous, etc. Alternatively, one of the substances may be removed from an individual's diet. It is also contemplated that one of the other substances, such as phenylalanine 12 or magnesium 14 may be added or removed depending upon their respective levels during the initial analysis.

The assessment of the modulated substances enables the provision of consulting/counseling services relative to the biological foundation for and between the substances associated with the affected substance, in the embodiment of calcium 10, phenylalanine 12 and magnesium 14, that affected substance being Tight Junction Protein 1 (TJP-1).

The relationships are studied using devices that will measure levels of the substances contained within the body that are listed above. The devices may be used to assess the corollary relationships between the substances in order to compare them to standard biodiagnostic baseline assessment.

The usage of these methods may also be used for assessment of homeostatic imbalances of relationships within modulated associated substances that can enable selection and administration of therapies to correct these disruptions instead of waiting to the point where mental or physical consequences will require expensive or sophisticated treatment of ailments or diseases that are the outcomes.

The usage of these methods may also be used for the design of kits, devices or systems to correlate the relationship between the various substances contained in the body as listed in this invention.

Kits of the present invention include one or more means for measurement of one or more of the substances or constituents which form the signaling molecule. Thus, as a nonlimiting example, a kit of the invention for the signaling molecule Tight Junction Protein 1 may comprise a means for measuring a level of calcium and/or a means for measuring a level of magnesium and/or a means for measuring a level of phenylalanine. Such kits may further comprise instructions for comparison of the measured levels and/or selecting and/or administering when necessary a treatment based upon the measured levels.

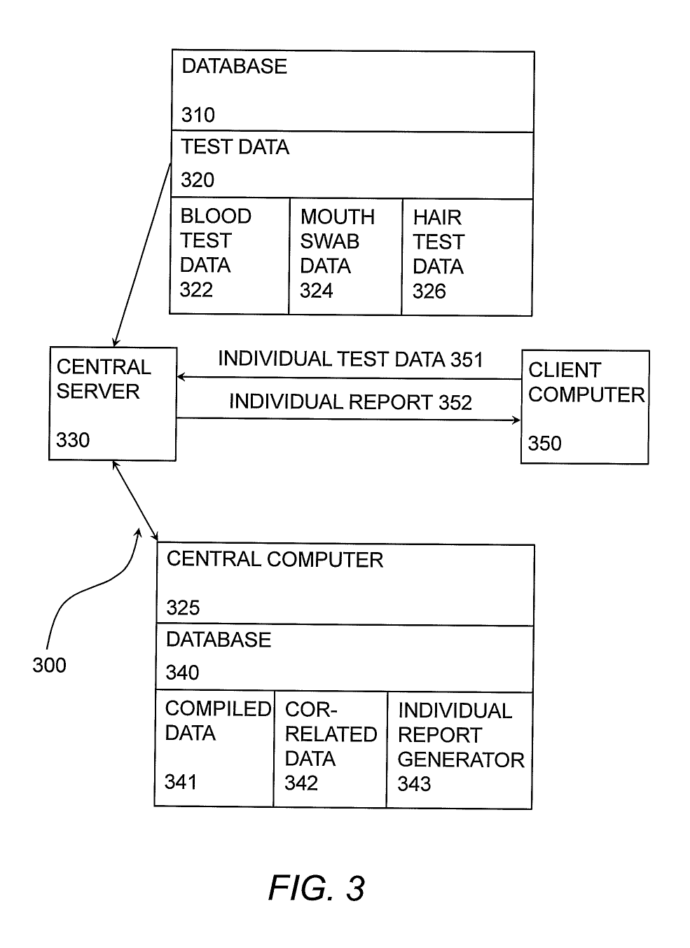

Devices can include, but should not be limited to, ones used within the veins and arteries, ongoing assessment as implanted devices, monitoring devices for home use or those used to perform diagnostic assessments in physician's offices, hospitals, diagnostic laboratories, home care or any other setting where the analysis of these substances contained in the blood or other specimens used for analysis are obtained for measurement of the levels and then compared to each other to perform assessments to maintain proper health and well being. A schematic of one embodiment of such a device or system is shown in FIG. 3.

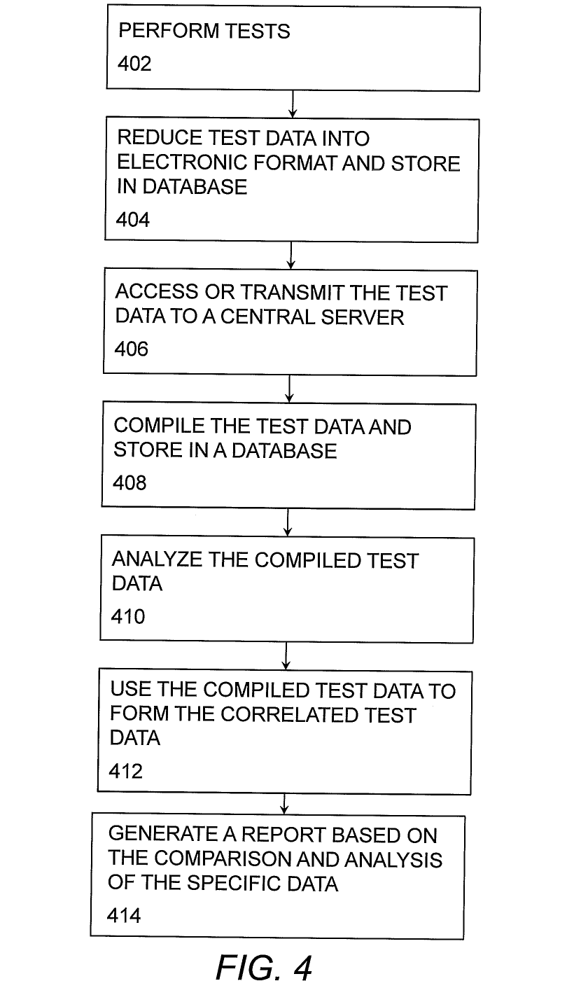

FIG. 3 shows a personalized report generator system 300 designed and arranged in accordance with an embodiment of the present invention. It should be understood that system 300 shows the basic components of the system and that more or less individualized components may be provided. FIG. 4 is a flow chart showing the steps involved in using the system 300.

The personalized report generator system 300 comprises a database 310 in which may be stored various test results. The data is stored in the database 310 as test data 320. The test data 320 is in a format that is capable of being manipulated by computers, processors, etc. The test data 320 may comprise various substance levels. In an exemplary embodiment of the present invention and shown in step 402 of FIG. 4, tests are performed on individual patients. In step 404, the results of these tests are then reduced into an electronic format in order to produce the test data 320. For example, the database 310 may store blood test data 322, mouth swab data 324 and hair test data 326. Other tests may include, but are not limited to, urine; fecal matter; collagen; chyle; interstitial fluid (tissue fluid); lymph; extracellular fluid; amniotic fluid; sweat tears; saliva; mucus; phlegm; hair; fingernails; bone marrow and breath. Each of these various tests may screen for a plurality of substances that are mentioned above in the list of modulated associated substances. As a nonlimiting example, the blood test data 322 may screen for calcium 10, phenylalanine 12 and magnesium 14.

The test data 320 may be organized and stored at the database 310. Alternatively, satellite locations may each maintain their own databases 310 that control their own records. In step 406, these locations may be accessed periodically by the central server 330 or the various databases 310 may transmit their results to a central location that is then accessible by a central server 330. Preferably the test data 320 is only accessed or transmitted when a certain threshold number of results are obtained. For example the test data 320 may not be sent to the central server 330 until at least one hundred tests are prepared to be transmitted.

In step 408, the transmitted test data 320 is compiled and stored as compiled test data 341. The central server 330 may keep the sent or accessed compiled test data 341 stored at its location in the central database 340, or the system 300 may provide a distributed storage system that stores the compiled test data 341 at external locations as well as the central database 340. However, it should be understood that when compiled test data 341 is stored at distributed sites, the system still functions as if they were stored at the central database 340. The compiled test data 341 may be stored so that the origin of the data is maintained in an anonymous fashion. In some instances tracking of the origin of a test result may only occur when a personalized report is requested.

In step 410, the compiled test data 341 is then analyzed in order to determine significant figures for conducting analysis. The analysis may be accomplished with the usage of a central computer(s) 325 of which the server 330 may be a part of, as well as the database 310. The analysis may be accomplished by statistically compiling and correlating the results for one specific test or by compiling the data for two separate items that are believed to have a relationship. Specific values from the plurality of compiled test data 341 having a similar feature may be analyzed. This may be accomplished through the application of known statistical algorithms. In step 412 of FIG. 4, the statistically compiled test data 341 may be used to form the correlated data 342. In step 414 of FIG. 4, the statistically correlated data 342 is then used by the individual report generator 343 to form the personalized medical prescription and diagnosis. Upon receipt of individual test data 351 from a client computer 350, the individual report generator 343 processes the data and transmits an individual report 352 to a client computer 350.

The compiled test data 341 may be periodically updated from the databases 310 and through the addition of more collected test data. The updating of the compiled test data 341 may provide additional information with which to refine the correlated data 342 and to refine the ranges used by the individual report generator 343 when producing the personalized medical prescription and diagnosis. Additionally the updating of the compiled test data 341 may be a continuous feature wherein the addition of new information is immediately reflected in the correlated data 342. This may provide a constant analysis of compiled test data 341 that may reflect changes in population and diets in society.

It should be understood that all modulated associated substances and/or signaling molecules disclosed above may be assessed, modulated and/or treated in an individual in the same fashion as that discussed with the nonlimiting exemplary first modulator, calcium 10, the nonlimiting exemplary second modulator, phenylalanine 12, and the nonlimiting exemplary third modulator, magnesium 14, in correlation with nonlimiting exemplary signaling molecule, tight junction protein 1 (TIP-1). Further, by the term “individual” when used herein, it is meant to include assessment, modulating and/or treatment in a whole subject such as a human or animal as well as at the cellular or subcellular level in humans and animals.

Another embodiment of the present invention thus relates to a method or kit for assessing, modulating and/or treating existing levels of the constituents necessary to form the signaling molecule tight junction protein 2 in an individual comprising: measuring a level of calcium; measuring a level of tyrosine; measuring a level of magnesium; comparing the measured levels; and selecting and/or administering when necessary a treatment based upon the measured levels.

Another embodiment of the present invention is a method or kit for assessing, modulating and/or treating existing levels of the constituents necessary to form the signaling molecule tight junction protein 3 in an individual comprising: measuring a level of calcium; measuring a level of tryptophan in the individual; measuring a level of magnesium; comparing the measured levels; and selecting and/or administering when necessary a treatment based upon the measured levels.

Another embodiment of the present invention is a method or kit for assessing, modulating and/or treating the existing levels of the constituents necessary to form the signaling molecule aldosterone in an individual comprising: measuring a level of sulfur; measuring a level of iron in the individual; measuring a level of nitric oxide; comparing the measured levels; and selecting and/or administering when necessary a treatment based upon the measured levels.

Another embodiment of the present invention is a method or kit for assessing, modulating and/or treating the existing levels of the constituents necessary to form the signaling molecule oxytocin in an individual comprising: measuring a level of sulfur; measuring a level of iron in the individual; measuring a level of arginine; comparing the measured levels; and selecting and/or administering when necessary a treatment based upon the measured levels.

Another embodiment of the present invention is a method or kit for assessing, modulating and/or treating the existing levels of the constituents necessary to form the signaling molecule serotonin in an individual comprising: measuring a level of sulfur; measuring a level of iron in the individual; measuring a level of histidine; comparing the measured levels; and selecting and/or administering when necessary a treatment based upon the measured levels.

Another embodiment of the present invention is a method or kit for assessing, modulating and/or treating the existing levels of the constituents necessary to form the signaling molecule adrenaline in an individual comprising: measuring a level of nickel; measuring a level of zinc in the individual; measuring a level of tryptophan; comparing the measured levels; and selecting and/or administering when necessary a treatment based upon the measured levels.

Another embodiment of the present invention is a method or kit for assessing, modulating and/or treating the existing levels of the constituents necessary to form the signaling molecule cortisol in an individual comprising: measuring a level of nickel; measuring a level of zinc in the individual; measuring a level of nitric oxide; comparing the measured levels; and selecting and/or administering when necessary a treatment based upon the measured levels.

Another embodiment of the present invention is a method or kit for assessing, modulating and/or treating the existing levels of the constituents necessary to form the signaling molecule p53 in an individual comprising: measuring a level of iron; measuring a level of sulfur in the individual; measuring a level of phosphorus; comparing the measured levels; and selecting and/or administering when necessary a treatment based upon the measured levels.

Another embodiment of the present invention is a method or kit for assessing, modulating and/or treating the existing levels of the constituents necessary to form the signaling molecule p63 in an individual comprising: measuring a level of copper; measuring a level of zinc in the individual; measuring a level of calcium; comparing the measured levels; and selecting and/or administering when necessary a treatment based upon the measured levels.

Another embodiment of the present invention is a method or kit for assessing, modulating and/or treating the existing levels of the constituents necessary to form the signaling molecule p73 in an individual comprising: measuring a level of nickel; measuring a level of zinc in the individual; measuring a level of boron; comparing the measured levels; and selecting and/or administering when necessary a treatment based upon the measured levels.

Another embodiment of the present invention is a method or kit for assessing, modulating and/or treating the existing levels of the constituents necessary to form the signaling molecule vitamin K2 in an individual comprising: measuring a level of phosphorus; measuring a level of calcium in the individual; measuring a level of valine; comparing the measured levels; and selecting and/or administering when necessary a treatment based upon the measured levels.

Another embodiment of the present invention is a method for assessing, modulating and/or treating the existing levels of the constituents necessary to form the signaling molecule vitamin K3 (a.k.a. MK4) in an individual comprising: measuring a level of phosphorus; measuring a level of calcium in the individual; measuring a level of leucine; comparing the measured levels; and selecting and/or administering when necessary a treatment based upon the measured levels.

Another embodiment of the present invention is a method or kit for assessing, modulating and/or treating the existing levels of the constituents necessary to form the signaling molecule Interleukin 15 in an individual comprising: measuring a level of phosphorus; measuring a level of calcium in the individual; measuring a level of nitric oxide; comparing the measured levels; and selecting and/or administering when necessary a treatment based upon the measured levels.

Another embodiment of the present invention is a method or kit for assessing, modulating and/or treating the existing levels of the constituents necessary to form the signaling molecule Interleukin 17 in an individual comprising: measuring a level of phosphorus; measuring a level of chromium in the individual; measuring a level of nitric oxide; comparing the measured levels; and selecting and/or administering when necessary a treatment based upon the measured levels.

Another embodiment of the present invention is a method or kit for assessing, modulating and/or treating the existing levels of the constituents necessary to form the signaling molecule nitric oxide synthase 3 in an individual comprising: measuring a level of bromine; measuring a level of iodine in the individual; measuring a level of phenylalanine; comparing the measured levels; and selecting and/or administering when necessary a treatment based upon the measured levels.

Another embodiment of the present invention is a method or kit for assessing, modulating and/or treating the existing levels of the constituents necessary to form the signaling molecule nitric oxide synthase 3 in an individual comprising: measuring a level of bromine; measuring a level of iodine in the individual; measuring a level of tyrosine; comparing the measured levels; and selecting and/or administering when necessary a treatment based upon the measured levels.

Another embodiment of the present invention is a method or kit for assessing, modulating and/or treating the existing levels of the constituents necessary to form the signaling molecule nitric oxide synthase 3 in an individual comprising: measuring a level of bromine; measuring a level of iodine in the individual; measuring a level of tryptophan; comparing the measured levels; and selecting and/or administering when necessary a treatment based upon the measured levels.

Another embodiment of the present invention is a method or kit for assessing, modulating and/or treating the existing levels of the constituents necessary to form the signaling molecule HMOX-1 in an individual comprising: measuring a level of iron; measuring a level of sulfur in the individual; measuring a level of threonine; comparing the measured levels; and selecting and/or administering when necessary a treatment based upon the measured levels.

Another embodiment of the present invention is a method or kit for assessing, modulating and/or treating the existing levels of the constituents necessary to form the signaling molecule HMOX-2 in an individual comprising: measuring a level of iron; measuring a level of sulfur in the individual; measuring a level of serine; comparing the measured levels; and selecting and/or administering when necessary a treatment based upon the measured levels.

Another embodiment of the present invention is a method or kit for assessing, modulating and/or treating the existing levels of the constituents necessary to form the signaling molecule HMOX-3 in an individual comprising: measuring a level of iron; measuring a level of sulfur in the individual; measuring a level of cysteine; comparing the measured levels; and selecting and/or administering when necessary a treatment based upon the measured levels.

Another embodiment of the present invention is a method or kit for assessing, modulating and/or treating the existing levels of the constituents necessary to form the signaling molecule TIG-1 in an individual comprising: measuring a level of phosphorus; measuring a level of calcium in the individual; measuring a level of phenylalanine; comparing the measured levels; and selecting and/or administering when necessary a treatment based upon the measured levels.

Another embodiment of the present invention is a method or kit for assessing, modulating and/or treating the existing levels of the constituents necessary to form the signaling molecule TIG-2 in an individual comprising: measuring a level of phosphorus; measuring a level of calcium in the individual; measuring a level of tyrosine; comparing the measured levels; and selecting and/or administering when necessary a treatment based upon the measured levels.

Another embodiment of the present invention is a method or kit for assessing, modulating and/or treating the existing levels of the constituents necessary to form the signaling molecule TIG-3 in an individual comprising: measuring a level of phosphorus; measuring a level of calcium in the individual; measuring a level of tryptophan; comparing the measured levels; and selecting and/or administering when necessary a treatment based upon the measured levels.

Another embodiment of the present invention is a method or kit for assessing, modulating and/or treating the existing levels of the constituents necessary to form the signaling molecule HER-1 in an individual comprising: measuring a level of phosphorus; measuring a level of chromium in the individual; measuring a level of tyrosine; comparing the measured levels; and selecting and/or administering when necessary a treatment based upon the measured levels.

Another embodiment of the present invention is a method or kit for assessing, modulating and/or treating the existing levels of the constituents necessary to form the signaling molecule HER-2 in an individual comprising: measuring a level of phosphorus; measuring a level of chromium in the individual; measuring a level of phenylalanine; comparing the measured levels; and selecting and/or administering when necessary a treatment based upon the measured levels.

Another embodiment of the present invention is a method or kit for assessing, modulating and/or treating the existing levels of the constituents necessary to form the signaling molecule HER-3 in an individual comprising: measuring a level of phosphorus; measuring a level of chromium in the individual; measuring a level of tryptophan; comparing the measured levels; and selecting and/or administering when necessary a treatment based upon the measured levels.

Another embodiment of the present invention is a method or kit for assessing, modulating and/or treating the existing levels of the constituents necessary to form the signaling molecule acetylcholinesterase in an individual comprising: measuring a level of phosphorus; measuring a level of calcium in the individual; measuring a level of histidine; comparing the measured levels; and selecting and/or administering when necessary a treatment based upon the measured levels.

Another embodiment of the present invention is a method or kit for assessing, modulating and/or treating the existing levels of the constituents necessary to form the signaling molecule butylcholinesterase in an individual comprising: measuring a level of phosphorus; measuring a level of calcium in the individual; measuring a level of arginine; comparing the measured levels; and selecting and/or administering when necessary a treatment based upon the measured levels.

Another embodiment of the present invention is a method or kit to assess, modulate and/or treat existing levels of the constituents necessary to form a signaling molecule for nucleotide activity comprising: measuring a level of phosphorus; measuring a level of histidine; measuring a level of sulfur; comparing the measured levels; and selecting and/or administering when necessary a treatment based upon the measured levels.

Another embodiment of the present invention is a method or kit to assess, modulate and/or treat existing levels of the constituents necessary to form a signaling molecule for nucleotide activity comprising: measuring a level of phosphorus; measuring a level of arginine; measuring a level of sulfur; comparing the measured levels; and selecting and/or administering when necessary a treatment based upon the measured levels.

Another embodiment of the present invention is a method or kit to assess, modulate and/or treat existing levels of the constituents necessary to form a signaling molecule for nucleotide activity comprising: measuring a level of phosphorous; measuring a level of histidine; measuring a level of silicon; comparing the measured levels; and selecting and/or administering when necessary a treatment based upon the measured levels.

Another embodiment of the present invention is a method or kit to assess, modulate and/or treat existing levels of the constituents necessary to form a signaling molecule for nucleotide activity comprising: measuring a level of phosphorous; measuring a level of arginine; measuring a level of silicon; comparing the measured levels; and selecting and/or administering when necessary a treatment based upon the measured levels.

Another embodiment of the present invention is a method or kit to assess, modulate and/or treat existing levels of the constituents that form a signaling molecules for nucleotide activity comprising: measuring a level of phosphorus; measuring a level of lysine; measuring a level of silicon; comparing the measured levels; and selecting and/or administering when necessary a treatment based upon the measured levels.

Another embodiment of the present invention is a method or kit to assess, modulate and/or treat existing levels of the constituents that form a signaling molecule for nucleotide activity comprising: measuring a level of phosphorus; measuring a level of threonine; measuring a level of silicon; comparing the measured levels; and selecting and/or administering when necessary a treatment based upon the measured levels.

Another embodiment of the present invention is a method or kit to assess, modulate and/or treat existing levels of the constituents that form a signaling molecule for nucleotide activity comprising: measuring a level of phosphorus; measuring a level of serine; measuring a level of silicon; comparing the measured levels; and selecting and/or administering when necessary a treatment based upon the measured levels.

Another embodiment of the present invention is a method or kit to assess, modulate and/or treat existing levels of the constituents that form a signaling molecule for nucleotide activity comprising: measuring a level of phosphorus; measuring a level of threonine; measuring a level of magnesium; comparing the measured levels; and selecting and/or administering when necessary a treatment based upon the measured levels.

Another embodiment of the present invention is a method or kit to assess, modulate and/or treat existing levels of the constituents that form a signaling molecule for nucleotide activity comprising: measuring a level of phosphorus; measuring a level of serine; measuring a level of magnesium; comparing the measured levels; and selecting and/or administering when necessary a treatment based upon the measured levels.

Another embodiment of the present invention is a method or kit to assess, modulate and/or treat existing levels of the constituents that form a signaling molecule for nucleotide activity comprising: measuring a level of phosphorus; measuring a level of histidine; measuring a level of magnesium; comparing the measured levels; and selecting and/or administering when necessary a treatment based upon the measured levels.

Another embodiment of the present invention is a method or kit to assess, modulate and/or treat existing levels of the constituents that form a signaling molecule for nucleotide activity comprising: measuring a level of phosphorus; measuring a level of arginine; measuring a level of magnesium; comparing the measured levels; and selecting and/or administering when necessary a treatment based upon the measured levels.

Another embodiment of the present invention is a method or kit to assess, modulate and/or treat existing levels of the constituents that form a signaling molecule for androstenone comprising: measuring a level of selenium; measuring a level of histidine; measuring a level of zinc; comparing the measured levels; and selecting and/or administering when necessary a treatment based upon the measured levels.

Another embodiment of the present invention is a method or kit to assess, modulate and/or treat existing levels of the constituents that form a signaling molecule for androstenediol comprising: measuring a level of selenium; measuring a level of arginine; measuring a level of zinc; comparing the measured levels; and selecting and/or administering when necessary a treatment based upon the measured levels.

Another embodiment of the present invention is a method or kit to assess, modulate and/or treat existing levels of the constituents that form a signaling molecule for androstenedione comprising: measuring a level of selenium; measuring a level of lysine; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule retinoblastoma-like protein (RBL) comprising: measuring a level of nickel; measuring a level of proline; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule retinoblastoma-like protein 1 (RBL-1) comprising: measuring a level of copper; measuring a level of proline; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule retinoblastoma-like protein 2 (RBL-2) comprising: measuring a level of iron; measuring a level of proline; measuring a level of manganese; comparing the measured levels; and selecting and/or administering when necessary a treatment based upon the measured levels.

Another embodiment of the present invention is a method or kit to assess, modulate and/or treat existing levels of the constituents that form the signaling molecule for Nerve Growth Factor (NGF) comprising: measuring a level of copper; measuring a level of glutamic acid; measuring a level of zinc; comparing the measured levels; and selecting and/or administering when necessary a treatment based upon the measured levels.

Another embodiment of the present invention is a method or kit to assess, modulate and/or treat existing levels of the constituents that form one of the signaling molecules known as a tyrosinase comprising: measuring a level of copper; measuring a level of phenylalanine; measuring a level of zinc; comparing the measured levels; and selecting and/or administering when necessary a treatment based upon the measured levels.

Another embodiment of the present invention is a method or kit to assess, modulate and/or treat existing levels of the constituents that form one of the signaling molecules known as a tyrosinase comprising: measuring a level of copper; measuring a level of tyrosine; measuring a level of zinc; comparing the measured levels; and selecting and/or administering when necessary a treatment based upon the measured levels.

Another embodiment of the present invention is a method or kit to assess, modulate and/or treat existing levels of the constituents that form one of the signaling molecules known as tyrosinase: measuring a level of copper; measuring a level of tryptophan; measuring a level of zinc; comparing the measured levels; and selecting and/or administering when necessary a treatment based upon the measured levels.

Another embodiment of the present invention is a method or kit to assess, modulate and/or treat existing levels of the constituents that form a signaling molecule for blood oxygenation comprising; measuring a level of sulfur; measuring a level of serine; measuring a level of iron; comparing the measured levels; and selecting and/or administering when necessary a treatment based upon the measured levels.

Another embodiment of the present invention is a method or kit to assess, modulate and/or treat existing levels of the constituents that form a signaling molecule for blood oxygenation comprising; measuring a level of iron; measuring a level of threonine; measuring a level of sulfur; comparing the measure levels; and selecting and/or administering when necessary a treatment based upon the measured levels.

Another embodiment of the present invention is a method or kit to assess, modulate and/or treat existing levels the constituents that form a signaling molecule for blood oxygenation comprising: measuring a level of iron; measuring a level of cysteine; measuring a level of sulfur; comparing the measured levels; and selecting and/or administering when necessary a treatment based upon the measured levels.

Another embodiment of the present invention is a method or kit to assess, modulate and/or treat existing levels of the constituents that form one of the signaling molecules for the Huntingtin gene comprising; measuring a level of phosphorus; measuring level of histidine; measuring a level of sulfur; comparing the measured levels; and selecting and/or administering when necessary a treatment based upon the measured levels.

Another embodiment of the present invention is a method or kit to assess, modulate and/or treat existing levels of the constituents that form one of the signaling molecules for the Huntingtin gene comprising; measuring the levels of phosphorus; measuring a level of arginine; measuring the levels of sulfur; comparing the measured levels; and selecting and/or administering when necessary a treatment based upon the measured levels.

Another embodiment of the present invention is a method or kit to assess, modulate and/or treat existing levels of the constituents that form one of the three forms of lactoferrin comprising: measuring a level of iron; measuring a level of glutamic acid; measuring a level of manganese; comparing the measured levels; and selecting and/or administering when necessary a treatment based upon the measured levels.

Another embodiment of the present invention is a method or kit to assess, modulate and/or treat existing levels of the constituents that form one of the three forms of lactoferrin comprising; measuring a level of iron; measuring a level of alanine; measuring a level of manganese; comparing the measured levels; and selecting and/or administering when necessary a treatment based upon the measured levels.

Another embodiment of the present invention is a method or kit to assess, modulate and/or treat existing levels of the constituents that form one of the three forms of lactoferrin comprising; measuring a level of iron; measuring a level of aspartic acid; measuring a level of manganese; comparing the measured levels; and selecting and/or administering when necessary a treatment based upon the measured levels.

Another embodiment of the present invention is a method or kit to assess, modulate and/or treat existing levels of the constituents that form one of the three forms of hololactoferrin comprising; measuring a level of iron; measuring a level of phenylalanine; measuring a level of manganese; comparing the measured levels; and selecting and/or administering when necessary a treatment based upon the measured levels.

Another embodiment of the present invention is a method or kit to assess, modulate and/or treat existing levels of the constituents that form one of the three forms of hololactoferrin comprising; measuring a level of iron; measuring a level of tyrosine; measuring a level of manganese; comparing the measured levels; and selecting and/or administering when necessary a treatment based upon the measured levels.

Another embodiment of the present invention is a method or kit to assess, modulate and/or treat existing levels of the constituents that form one of the three forms of hololactoferrin comprising; measuring a level of iron; measuring a level of tryptophan; measuring a level of manganese; comparing the measured levels; and selecting and/or administering when necessary a treatment based upon the measured levels.

Another embodiment of the present invention is a method or kit to assess, modulate and/or treat existing levels of the constituents that form one of the three forms of apolactoferrin comprising: measuring a level of iron; measuring a level of histidine; measuring a level of manganese; comparing the measured levels; and selecting and/or administering when necessary a treatment based upon the measured levels.

Another embodiment of the present invention is a method or kit to assess, modulate and/or treat existing levels of the constituents that form another of the three forms of apolactoferrin comprising: measuring a level of iron; measuring a level of arginine; measuring a level of manganese; comparing the measured levels; and selecting and/or administering when necessary a treatment based upon the measured levels.

Another embodiment of the present invention is a method or kit assess, modulate and/or treat existing levels of the constituents that form another of the three forms of apolactoferrin comprising: measuring a level of iron; measuring a level of lysine; measuring a level of manganese; comparing the measured levels; and selecting and/or administering when necessary a treatment based upon the measured levels.

Another embodiment of the present invention is a method or kit to assess, modulate and/or treat existing levels of the constituents that form the signaling molecule Interleukin 1 (IL-1) comprising: measuring a level of aluminum; measuring a level of nitric oxide; measuring a level of iron; comparing the measured levels; and selecting and/or administering when necessary a treatment based upon the measured levels.

Another embodiment of the present invention is a method or kit to assess, modulate and/or treat existing levels of the constituents that fat m the signaling molecule Interleukin 3 (IL-3) comprising: measuring a level of calcium; measuring a level of nitric oxide; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule Interleukin 5 (IL-5) comprising: measuring a level of selenium; measuring a level of nitric oxide; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule Interleukin 8 (IL-8) comprising: measuring a level of selenium; measuring a level of zinc; measuring a level of iodine; comparing the measured levels; and selecting and/or administering when necessary a treatment based upon the measured levels.

Another embodiment of the present invention is a method or kit to assess, modulate and/or treat existing levels of the constituents that form the signaling molecule Interleukin 13 (IL-13) comprising: measuring a level of iron; measuring a level of nitric oxide; measuring a level of manganese; comparing the measured levels; and selecting and/or administering when necessary a treatment based upon the measured levels.

Another embodiment of the present invention is a method or kit to assess, modulate and/or treat existing levels of the constituents that form the signaling molecule Interleukin 16 (IL-16) comprising: measuring a level of sulfur; measuring a level of nitric oxide; measuring a level of iron; comparing the measured levels; and selecting and/or administering when necessary a treatment based upon the measured levels.

Another embodiment of the present invention is a method or kit to assess, modulate and/or treat existing levels of the constituents that form the signaling molecule Interleukin 18 (IL-18) comprising: measuring a level of copper; measuring a level of nitric oxide; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule Interleukin 23 (IL-23) comprising: measuring a level of bromine; measuring a level of nitric oxide; measuring a level of iodine; comparing the measured levels; and selecting and/or administering when necessary a treatment based upon the measured levels.

Another embodiment of the present invention is a method or kit to assess, modulate and/or treat existing levels of the constituents that form the signaling molecule Interleukin 32 (IL-32) comprising: measuring a level of calcium; measuring a level of nitric oxide; measuring a level of magnesium; comparing the measured levels; and selecting and/or administering when necessary a treatment based upon the measured levels.

Another embodiment of the present invention is a method or kit to assess, modulate and/or treat existing levels of the constituents that form the signaling molecule Interferon gamma (IFN-g) comprising: measuring a level of iron; measuring a level of hydrogen sulfide; measuring a level of manganese; comparing the measured levels; and selecting and/or administering when necessary a treatment based upon the measured levels.

Another embodiment of the present invention is a method or kit to assess, modulate and/or treat existing levels of the constituents that form the signaling molecule retinoblastoma protein (pRB) comprising: measuring a level of copper; measuring a level of hydrogen sulfide; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule retinoblastoma protein 1 (pRB-1) comprising: measuring a level of nickel; measuring a level of hydrogen sulfide; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule troponin T comprising; measuring a level of calcium; measuring a level of phenylalanine; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule troponin C comprising: measuring a level of calcium; measuring a level of tyrosine; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule troponin I comprising: measuring a level of calcium; measuring a level of tryptophan; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule which is one of the three sub-units of troponin TNT comprising: measuring a level of calcium; measuring a level of histidine; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule which is one of the three sub-units of troponin TNT comprising: measuring a level of calcium; measuring a level of arginine; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule which is one of the three sub-units of troponin TNT comprising: measuring a level of calcium; measuring a level of lysine; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule which is one of the three sub-units of troponin TNC comprising: measuring a level of calcium; measuring a level of leucine; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule which is one of the three sub-units of troponin TNC comprising: measuring a level of calcium; measuring a level of isoleucine; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule which is one of the three sub-units of troponin TNC comprising: measuring a level of calcium; measuring a level of valine; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule Lefty 1 comprising: measuring a level of calcium; measuring a level of serine; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule Lefty 2 comprising: measuring a level of calcium; measuring a level of cysteine; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule NODAL comprising: measuring a level of calcium; measuring a level of threonine; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule SynCAM2 comprising: measuring a level of calcium; measuring a level of threonine; measuring a level of magnesium; comparing the measured levels; and selecting and/or administering when necessary a treatment based upon the measured levels.

Another embodiment of the present invention is a method or kit to assess, modulate and/or treat existing levels of the constituents that form the signaling molecule SynCAM1 comprising: measuring a level of calcium; measuring a level of serine; measuring a level of magnesium; comparing the measured levels; and selecting and/or administering when necessary a treatment based upon the measured levels.

Another embodiment of the present invention is a method or kit to assess, modulate and/or treat existing levels of the constituents that form the signaling molecule SynCAM3 comprising: measuring a level of calcium; measuring a level of cysteine; measuring a level of magnesium; comparing the measured levels; and selecting and/or administering when necessary a treatment based upon the measured levels.

Another embodiment of the present invention is a method or kit to assess, modulate and/or treat existing levels of the constituents that form the signaling molecule T4 thyroid hormone comprising: measuring a level of bromine; measuring a level of serine; measuring a level of iodine; comparing the measured levels; and selecting and/or administering when necessary a treatment based upon the measured levels.

Another embodiment of the present invention is a method or kit to assess, modulate and/or treat existing levels of the constituents that form the signaling molecule T3 thyroid hormone comprising: measuring a level of bromine; measuring a level of cysteine; measuring a level of iodine; comparing the measured levels; and selecting and/or administering when necessary a treatment based upon the measured levels.

Another embodiment of the present invention is a method or kit to assess, modulate and/or treat existing levels of the constituents that form the signaling molecule T2 thyroid hormone comprising: measuring a level of bromine; measuring a level of threonine; measuring a level of iodine; comparing the measured levels; and selecting and/or administering when necessary a treatment based upon the measured levels.

Another embodiment of the present invention is a method or kit to assess, modulate and/or treat existing levels of the constituents that form signaling molecule secretin comprising; measuring a level of bromine; measuring a level of glutamic acid; measuring a level of iodine; comparing the measured levels; and selecting and/or administering when necessary a treatment based upon the measured levels.

Another embodiment of the present invention is a method or kit to assess, modulate and/or treat existing levels of the constituents that form the signaling molecule gastric inhibitory peptide (GIP) comprising; measuring a level of bromine; measuring a level of aspartic acid; measuring a level of iodine; comparing the measured levels; and selecting and/or administering when necessary a treatment based upon the measured levels.

Another embodiment of the present invention is a method or kit to assess, modulate and/or treat existing levels of the constituents that form the signaling molecule vasoactive intestinal peptide (VIP) comprising; measuring a level of bromine; measuring a level of alanine; measuring a level of iodine; comparing the measure levels; and selecting and/or administering when necessary a treatment based upon the measured levels.

Another embodiment of the present invention is a method or kit to assess, modulate and/or treat existing levels of the constituents that form the signaling molecule alpha aminobutyric acid comprising; measuring a level of iron; measuring a level of glutamic acid; measuring a level of sulfur; comparing the measured levels; and selecting and/or administering when necessary a treatment based upon the measured levels.

Another embodiment of the present invention is a method or kit to assess, modulate and/or treat existing levels of the constituents that form the signaling molecule beta aminobutyric acid comprising; measuring a level of iron; measuring a level of aspartic acid; measuring a level of sulfur; comparing the measure levels; and selecting and/or administering when necessary a treatment based upon the measured levels.

Another embodiment of the present invention is a method or kit to assess, modulate and/or treat existing levels of the constituents that form the signaling molecule gamma aminobutyric acid comprising; measuring a level of iron; measuring a level of alanine; measuring a level of sulfur; comparing the measured levels; and selecting and/or administering when necessary a treatment based upon the measured levels.

Another embodiment of the present invention is a method or kit to assess, modulate and/or treat existing levels of the constituents that form the signaling molecule Nuclear Respiratory Factor 1 (NRF-1) comprising; measuring a level of selenium; measuring a level of leucine; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule Nuclear Respiratory Factor 2 (NRF-2) comprising; measuring a level of selenium; measuring a level of isoleucine; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule Nuclear Respiratory Factor 3 (NRF-3) comprising; measuring a level of selenium; measuring a level of valine; measuring a level of zinc; comparing the measured levels; and selecting and/or administering when necessary a treatment based upon the measured levels.

Another embodiment of the present invention is a method or kit to assess, modulate and/or treat existing levels of the constituents that form the signaling molecule NPY-PYY-PP comprising: measuring a level of Interleukin 12; measuring a level of Interleukin 16; measuring a level of Interleukin 18; comparing the measured levels; and selecting and/or administering when necessary a treatment based upon the measured levels.

Another embodiment of the present invention is a method or kit to assess, modulate and/or treat existing levels of the constituents that form signaling molecule Caspase 1 comprising: measuring a level of iron; measuring a level of threonine; measuring a level of manganese; comparing the measured levels; and selecting and/or administering when necessary a treatment based upon the measured levels.

Another embodiment of the present invention is a method or kit to assess, modulate and/or treat existing levels of the constituents that form signaling molecule Caspase 2 comprising: measuring a level of iron; measuring a level of serine; measuring a level of manganese; comparing the measured levels; and selecting and/or administering when necessary a treatment based upon the measured levels.

Another embodiment of the present invention is a method or kit to assess, modulate and/or treat existing levels of the constituents that form the signaling molecule Caspase 3 comprising: measuring a level of iron; measuring a level of cysteine; measuring a level of manganese; comparing the measured levels; and selecting and/or administering when necessary a treatment based upon the measured levels.

Another embodiment of the present invention is a method or kit to assess, modulate and/or treat existing levels of the constituents that form the signaling molecule nitrogenase comprising: measuring a level of iron; measuring a level of sulfur; measuring a level of molybdenum; comparing the measured levels; and selecting and/or administering when necessary a treatment based upon the measured levels.

Another embodiment of the present invention is a method or kit to assess, modulate and/or treat existing levels of the constituents that form the signaling molecule DISC 1 comprising: measuring a level of sodium; measuring a level of proline; measuring a level of potassium; comparing the measured levels; and selecting and/or administering when necessary a treatment based upon the measured levels.

Another embodiment of the present invention is a method or kit to assess, modulate and/or treat existing levels of the constituents that form the signaling molecule DISC 2 comprising: measuring a level of sodium; measuring a level of glutamine; measuring a level of potassium; comparing the measured levels; and selecting and/or administering when necessary a treatment based upon the measured levels.

Another embodiment of the present invention is a method or kit to assess, modulate and/or treat existing levels of the constituents that form the signaling molecule TSNAX comprising: measuring a level of sodium; measuring a level of glycine; measuring a level of potassium; comparing the measured levels; and selecting and/or administering when necessary a treatment based upon the measured levels.

Another embodiment of the present invention is a method or kit to assess, modulate and/or treat existing levels of the constituents that form the signaling molecule lipoprotein receptor related protein 4 (LRP-4) comprising: measuring a level of sodium; measuring a level of serine; measuring a level of potassium; comparing the measured levels; and selecting and/or administering when necessary a treatment based upon the measured levels.

Another embodiment of the present invention is a method or kit to assess, modulate and/or treat existing levels of the constituents that form the signaling molecule lipoprotein receptor related protein 5 (LRP-5) comprising: measuring a level of sodium; measuring a level of threonine; measuring a level of potassium; comparing the measured levels; and selecting and/or administering when necessary a treatment based upon the measured levels.

Another embodiment of the present invention is a method or kit to assess, modulate and/or treat existing levels of the constituents that form the signaling molecule lipoprotein receptor related protein 6 (LRP-6) comprising: measuring a level of sodium; measuring a level of cysteine; measuring a level of potassium; comparing the measured levels; and selecting and/or administering when necessary a treatment based upon the measured levels.

Another embodiment of the present invention is a method or kit to assess, modulate and/or treat existing levels of the constituents that form the signaling molecule ceruloplasmin comprising: measuring a level of copper; measuring a level of zinc; measuring a level of iron; comparing the measured levels; and selecting and/or administering when necessary a treatment based upon the measured levels.

Another embodiment of the present invention is a method or kit for assessing, modulating and/or treating existing levels of the constituents that form the signaling molecule Interleukin 2 comprising: measuring a level of sodium; measuring a level of potassium; measuring a level of chloride; comparing the measured levels; and selecting and/or administering when necessary a treatment based upon the measured levels.

Another embodiment of the present invention is a method of kit for assessing, modulating and/or treating the existing levels of the constituents that form the signaling molecule Interleukin 4 comprising: measuring a level of nickel; measuring a level of zinc; measuring a level of boron; comparing the measured levels; and selecting and/or administering when necessary a treatment based upon the measured levels.

Another embodiment of the present invention is a method or kit for assessing, modulating and/or treating the existing levels of the constituents that form the signaling molecule Interleukin 11 comprising: measuring a level of phosphorus; measuring a level of calcium; measuring a level of magnesium; comparing the measured levels; and selecting and/or administering when necessary a treatment based upon the measured levels.

Another embodiment of the present invention is a method or kit for assessing, modulating and/or treating the existing levels of the constituents that form the signaling molecule Interleukin 12 comprising: measuring a level of nickel; measuring a level of zinc; measuring a level of nitric oxide; comparing the measured levels; and selecting and/or administering when necessary a treatment based upon the measured levels.

Another embodiment of the present invention is a method or kit for assessing, modulating and/or treating the existing levels of the constituents that form the signaling molecule Interleukin 33 comprising: measuring a level of sodium; measuring a level of potassium; measuring a level of nitric oxide; comparing the measured levels; and selecting and/or administering when necessary a treatment based upon the measured levels.

Another embodiment of the present invention is a method or kit for assessing, modulating and/or treating the existing levels of the constituents that form one of the three subunits of TNI comprising measuring a level of calcium; measuring a level of zinc; measuring a level of glutamic acid; comparing the measured levels; and selecting and/or administering when necessary a treatment based upon the measured levels.

Another embodiment of the present invention is a method or kit for assessing, modulating and/or treating the existing levels of the constituents that form one of the three subunits of TNI comprising: measuring a level of calcium; measuring a level of zinc; measuring a level of alanine; comparing the measured levels; and selecting and/or administering when necessary a treatment based upon the measured levels.

Another embodiment of the present invention is a method or kit for assessing, modulating and/or treating the existing levels of the constituents that form one of the three subunits of TNI comprising: measuring a level of calcium; measuring a level of zinc; measuring a level of aspartic acid; comparing the measured levels; and selecting and/or administering when necessary a treatment based upon the measured levels.

Another embodiment of the present invention is a method and kit for assessing, modulating and/or treating the homeostatic relationships between the levels of the neurohormones aldosterone, oxytocin, serotonin, adrenaline and cortisol comprising: measuring the levels of these neurohormones; comparing the measured levels; and selecting and/or administering when necessary a treatment based upon the measured levels.

Still another embodiment of the present invention is a method and kit for assessing, modulating and/or treating the homeostatic relationships between the levels of the neurohormones norepinephrine, prolactin and dopamine comprising: measuring the levels of these neurohormones; comparing the measured levels; and selecting and/or administering when necessary a treatment based upon the measured levels.

In all of the above embodiments, in addition to assessing levels of each constituent forming the singling molecule, the method may further comprise modulating a level of one or more constituents so that formation of the signaling molecule is increased or decreased. Further, the method may comprise selecting and, when needed, administering a selected treatment to alter a level of one or more constituents thereby establishing homeostasis of the affected signaling molecule. Such treatments may thus comprise, but are in no way limited to, administering one or more of the measured substances and/or an agent which alters levels of one or more of the measured substances or a dietary change which alters levels of one or more of the measured substances.

It is to be understood that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

What is claimed is:

1. A method for providing homeostasis of signaling molecule in an individual via the levels of modulators comprising:
measuring a level of a first modulator;
measuring a level of second modulator;
measuring a level of third modulator;
comparing the measured modulator levels; and
establishing homeostasis of the signaling molecule based upon the measured modulator levels.

2. The method of claim 1 wherein the first modulator is calcium, the second modulator is phenylalanine, tyrosine or tryptophan the third modulator is magnesium and the signaling molecule is tight junction protein-1, tight junction protein-2 or tight junction protein-3.

3. The method of claim 1 wherein the first modulator is phosphorus, the second modulator is sulfur, silicon or magnesium, the third modulator is histidine, arginine, lysine, threonine, or serine and the signaling molecules are for nucleotide activity.

4. The method of claim 1 wherein the first modulator is selenium, the second modulator is zinc, the third modulator is histidine and the signaling molecule is androstenone.

5. The method of claim 1 wherein the first modulator is selenium, the second modulator is zinc, the third modulator is arginine and the signaling molecule is androstenediol.

6. The method of claim 1 wherein the first modulator is selenium, the second modulator is zinc, the third modulator is lysine and the signaling molecule is androstendione.

7. The method of claim 1 wherein the first modulator is nickel, copper or iron, the second modulator is zinc or manganese, the third modulator is proline and the signaling molecule is retinoblastoma-like protein. retinoblastoma-like protein 1 or retinoblastoma-like protein 2.

8. The method of claim 1 wherein the first modulator is copper, the second modulator is zinc, the third modulator is glutamic acid and the signaling molecule is Nerve Growth Factor.

9. The method of claim 1 wherein the first modulator is copper, the second modulator is zinc, the third modulator is phenylalanine, tyrosine or tryptophan and the signaling molecules are PTEN, ETS1 and ETS2.

10. The method of claim 1 wherein the first modulator is sulfur, the second modulator is iron, the third modulator is serine, threonine or cysteine and the signaling molecules are for blood oxygenation.

11. The method of claim 1 wherein the first modulator is phosphorus, the second modulator is sulfur, the third modulator is histidine or arginine and the signaling molecules are for the Huntingtin gene.

12. The method of claim 1 wherein the first modulator is iron, the second modulator is manganese, the third modulator is glutamic acid, alanine or aspartic acid and the signaling molecules are the three forms of lactoferrin.

13. The method of claim 1 wherein the first modulator is iron, the second modulator is manganese, the third modulator is phenylalanine, tyrosine or tryptophan and the signaling molecules are the three forms of hololactoferrin.

14. The method of claim 1 wherein the first modulator is iron, the second modulator is manganese, the third modulator is histidine, arginine, or lysine and the signaling molecules are the three forms of apolactoferrin.

15. The method of claim 1 wherein the first modulator is aluminum, the second modulator is iron, the third modulator is nitric oxide and the signaling molecule is interleukin 1.

16. The method of claim 1 wherein the first modulator is calcium, the second modulator is zinc, the third modulator is nitric oxide and the signaling molecule is interleukin 3.

17. The method of claim 1 wherein the first modulator is selenium, the second modulator is zinc, the third modulator is nitric oxide and the signaling molecule is interleukin 5.

18. The method of claim 1 wherein the first modulator is selenium, the second modulator is zinc, the third modulator is iodine and the signaling molecule is interleukin 8.

19. The method of claim 1 wherein the first modulator is iron, the second modulator is manganese, the third modulator is nitric oxide and the signaling molecule is interleukin 13.

20. The method of claim 1 wherein the first modulator is sulfur, the second modulator is iron, the third modulator is nitric oxide and the signaling molecule is interleukin 16.

21. The method of claim 1 wherein the first modulator is copper, the second modulator is zinc, the third modulator is nitric oxide and the signaling molecule is interleukin 18.

22. The method of claim 1 wherein the first modulator is bromine, the second modulator is iodine, the third modulator is nitric oxide and the signaling molecule is interleukin 23.

23. The method of claim 1 wherein the first modulator is calcium, the second modulator is magnesium, the third modulator is nitric oxide and the signaling molecule is interleukin 32.

24. The method of claim 1 wherein the first modulator is iron, the second modulator is manganese, the third modulator is hydrogen sulfide and the signaling molecule is interferon gamma.

25. The method of claim 1 wherein the first modulator is copper or nickel, the second modulator is zinc, the third modulator is hydrogen sulfide and the signaling molecule is retinoblastoma protein or retinoblastoma protein 1.

26. The method of claim 1 wherein the first modulator is calcium, the second modulator is zinc, the third modulator is phenylalanine, tyrosine or tryptophan and the signaling molecule is troponin T, troponin C or troponin I.

27. The method of claim 1 wherein the first modulator is calcium, the second modulator is zinc, the third modulator is histidine, arginine or lysine and the signaling molecules are the three subunits of troponin TNT.

28. The method of claim 1 wherein the first modulator is calcium, the second modulator is zinc, the third modulator is leucine, isoleucine or valine and the signaling molecules are the three subunits of troponin TNC.

29. The method of claim 1 wherein the first modulator is calcium, the second modulator is zinc, the third modulator is serine or cysteine and the signaling molecule is Lefty 1 or Lefty 2.

30. The method of claim 1 wherein the first modulator is calcium, the second modulator is zinc, the third modulator is threonine and the signaling molecule is Nodal.

31. The method of claim 1 wherein the first modulator is calcium, the second modulator is magnesium, the third modulator is threonine, serine or cysteine and the signaling molecule is SynCAM2, SynCAM1 or SynCAM3.

32. The method of claim 1 wherein the first modulator is bromine, the second modulator is iodine, the third modulator is serine, cysteine or threonine and the signaling molecule is T4 thyroid hormone, T3 thyroid hormone or T2 thyroid hormone.

33. The method of claim 1 wherein the first modulator is bromine, the second modulator is iodine, the third modulator is glutamic acid and the signaling molecule is secretin.

34. The method of claim 1 wherein the first modulator is bromine, the second modulator is iodine, the third modulator is aspartic acid and the signaling molecule is gastric inhibitory peptide.

35. The method of claim 1 wherein the first modulator is bromine, the second modulator is iodine, the third modulator is alanine and the signaling molecule is vasoactive intestinal peptide.

36. The method of claim 1 wherein the first modulator is iron, the second modulator is sulfur, the third modulator is glutamic acid, aspartic acid or alanine and the signaling molecule is alpha aminobutyric acid, beta aminobutyric acid or gamma aminobutyric acid.

37. The method of claim 1 wherein the first modulator is selenium, the second modulator is zinc, the third modulator is leucine, isoleucine or valine and the signaling molecule is Nuclear Respiratory Factor 1, Nuclear Respiratory Factor 2 or Nuclear Respiratory Factor 3.

38. The method of claim 1 wherein the first modulator is interleukin 12, the second modulator is interleukin 18, the third modulator is interleukin 16 and the signaling molecule is NPY-PYY-PP.

39. The method of claim 1 wherein the first modulator is iron, the second modulator is manganese, the third modulator is threonine, serine or cysteine and the signaling molecule is caspase 1, caspase 2 or caspase 3.

40. The method of claim 1 wherein the first modulator is iron, the second modulator is sulfur, the third modulator is molybdenum and the signaling molecule is nitrogenase.

41. The method of claim 1 wherein the first modulator is sodium, the second modulator is potassium, the third modulator is proline or glutamine and the signaling molecule is DISC 1 or DISC 2.

42. The method of claim 1 wherein the first modulator is sodium, the second modulator is potassium, the third modulator is glycine and the signaling molecule is TSNAX.

43. The method of claim 1 wherein the first modulator is sodium, the second modulator is potassium, the third modulator is serine, threonine or cysteine and the signaling molecule is lipoprotein related receptor protein 4, lipoprotein related receptor protein 5 or lipoprotein related receptor protein 6.

44. The method of claim 1 wherein the first modulator is copper, the second modulator is zinc, the third modulator is iron and the signaling molecule is ceruloplasmin.

45. The method of claim 1 wherein the first modulator is sulfur, the second modulator is nitric oxide, the third modulator is iron and the signaling molecule is a neurohormone aldosterone.

46. The method of claim 1 wherein the first modulator is sulfur, the second modulator is arginine, the third modulator is iron and the signaling molecule is a neurohormone oxytocin.

47. The method of claim 1 wherein the first modulator is sulfur, the second modulator is histidine, the third modulator is iron and the signaling molecule is a neurohormone serotonin.

48. The method of claim 1 wherein the first modulator is zinc, the second modulator is tryptophan, the third modulator is nickel and the signaling molecule is a neurohormone adrenaline.

49. The method of claim 1 wherein the first modulator is zinc, the second modulator is nitric oxide, the third modulator is nickel and the signaling molecule is a neurohormone cortisol.

50. The method of claim 1 wherein the first modulator is iron, the second modulator is phosphorus, the third modulator is sulfur and the signaling molecule is p53.

51. The method of claim 1 wherein the first modulator is copper, the second modulator is zinc, the third modulator is calcium and the signaling molecule is p63.

52. The method of claim 1 wherein the first modulator is nickel, the second modulator is zinc, the third modulator is boron and the signaling molecule is p73.

53. The method of claim 1 wherein the first modulator is phosphorus, the second modulator is leucine or valine, the third modulator is calcium and the signaling molecule is vitamin K2 or vitamin K3.

54. The method of claim 1 wherein the first modulator is phosphorus, the second modulator is nitric oxide, the third modulator is calcium or chromium and the signaling molecule is Interleukin-15 or Interleukin-17.

55. The method of claim 1 wherein the first modulator is bromine, the second modulator is phenylalanine, tyrosine or tryptophan, the third modulator is iodine and the signaling molecules are the three forms of nitric oxide synthase 3.

56. The method of claim 1 wherein the first modulator is iron, the second modulator is threonine, serine or cystein the third modulator is sulfur and the signaling molecule is HMOX1, HMOX2 or HMOX3.

57. The method of claim 1 wherein the first modulator is phosphorus, the second modulator is phenylalanine, tyrosine or tryptophan the third modulator is calcium and the signaling molecule is TIG-1, TIG-2 or TIG-3.

58. The method of claim 1 wherein the first modulator is phosphorus, the second modulator is tyrosine, phenylalanine or tryptophan the third modulator is chromium and the signaling molecule is HER-1, HER-2 or HER-3.

59. The method of claim 1 wherein the first modulator is phosphorus, the second modulator is histidine or arginine, the third modulator is calcium and the signaling molecule is acetylcholinesterase or butylcholinesterase.

60. The method of claim 1 wherein the first modulator is calcium, the second modulator is zinc, the third modulator is glutamic acid, alanine or aspartic acid and the signaling molecules are the three subunits of troponin TNI.

61. The method of claim 1 wherein the first modulator is sodium, the second modulator is potassium, the third modulator is chloride and the signaling molecule is Interleukin 2 (IL-2).

62. The method of claim 1 wherein the first modulator is phosphorus, the second modulator is calcium, the third modulator is magnesium and the signaling molecule is Interleukin 11 (IL-11).

63. The method of claim 1 wherein the first modulator is nickel, the second modulator is zinc, the third modulator is boron and the signaling molecule is Interleukin 12 (IL-4).

64. The method of claim 1 wherein the first modulator is nickel, the second modulator is zinc, the third modulator is nitric oxide and the signaling molecule is Interleukin 12 (IL-12).

65. The method of claim 1 wherein the first modulator is sodium, the second modulator is potassium, the third modulator is nitric oxide and the signaling molecule is Interleukin 33 (IL-33).

66. The method of claim 1, further comprising administering the first modulator, the second modulator, or the third modulator to the individual.

67. The method of claim 1, further comprising reducing intake of the first modulator, the second modulator or the third modulator by the individual.

68. The method of claim 1, wherein test data to which measured modulator levels are compared is stored and compiled in a central computer.

69. The method of claim 1, wherein the levels of the first, second and third modulators are re-measured.

70. A method for providing homeostasis of neurohormones aldosterone, oxytocin, serotonin, adrenaline and cortisol comprising: measuring the levels of neurohormones aldosterone, oxytocin, serotonin, adrenaline and cortisol; comparing the measured levels; and selecting and/or administering when necessary a treatment based upon the measured levels.

71. A method for providing homeostasis of neurohormones norepinephrine, prolactin and dopamine comprising: measuring the levels of neurohormones norepinephrine, prolactin and dopamine; comparing the measured levels; and selecting and/or administering when necessary a treatment based upon the measured levels.

72. A kit for assessing homeostasis of signaling molecule in an individual via levels of modulators and/or constituents forming the signaling molecule, said kit comprising:
a means for measuring a level of a first modulator and/or constituent;
a means for measuring a level of second modulator and/or constituent; and
a means for measuring a level of third modulator and/or constituent.
